(19) United States
(12) Patent Application Publication
(10) Pub. No.: US 2021/0055283 A1
(43) Pub. Date: Feb. 25, 2021

(54) Automated 2-D/3-D Cells, Organs, Human Culture Devices with Multimodal Activation and Monitoring

(71) Applicant: John Collins, Irvine, CA (US)

(72) Inventor: John Collins, Irvine, CA (US)

(21) Appl. No.: 15/917,577

(22) Filed: Mar. 10, 2018

(51) Int. Cl. G01N 33/50; C12M 1/34; C12M 1/32; C12M 3/06; C12M 1/00; C12M 1/42

(52) U.S. Cl. CPC G01N 33/5088; C12M 41/46; C12M 23/12; C12M 23/16; C12M 35/04; C12M 29/00; C12M 29/10; C12M 35/02; C12M 23/40

(57) ABSTRACT

There is provided systems and methods for performing fluidic perfusion, recirculation and interacting organ in standard wells or microfluidic reactors loading cells or organs into an insert or chip. The perfusion system can provide new media to the cell or organs while the circulation system can provide convective mixing of fluids within a well or between one or more organs in an assay. The system can be placed in an incubator or microscope and perform multimodal stimulation and sensing. The system includes electromechanical control, microfluidic lid and inserts or chips for performing automated cell based assay, organ of a chip or human on a chip in a remote-controlled environment.

### CROSS-REFERENCE TO RELATED APPLICATIONS

The present application claims the benefit of and priority to U.S. Provisional Patent Application titled “Automated 2-D/3-D Cells, Organs, Human Culture Devices with Multimodal Activation and Monitoring system,” Ser. No. 62/469,526, filed on Mar. 10, 2017. The disclosure in this provisional application is hereby incorporated fully by reference into the present application.

### STATEMENT REGARDING FEDERALLY-SPONSORED RESEARCH AND DEVELOPMENT

This invention was made with Government support under contract No. R43HL118938 and R43MH104170 awarded by the National Institute of Health (NIH). The Government has certain rights in this invention.

### FIELD OF THE INVENTION

The present invention generally relates to cells, organs and human culture devices and methods, and more particularly to systems and methods for multiplexed cell based assays in good laboratory practice.

### BACKGROUND OF THE INVENTION

Microfluidic systems provide remarkable features for controlling fluidics in cell, organ and human assays. Fluidic addition or removal or mix of two or more reagents, develop multiple composition of reagents, perform concentration gradient and periodic delivery of fluids. Monitoring systems probe cellular systems for growth or signaling due to activation parameters not limited to optical, electrical, mechanical, chemical and acoustics.

### SUMMARY

The present invention is directed to a system and method for multiple organs based assays using microfluidic system equipped with fluidic operations such as perfusion and recirculation, cell/organ stimulation using optical, chemical, mechanical, acoustics and electrical, cell/organ monitoring using optical imaging, electrical field potentials, electrical impedance and cell/organ media monitoring using pH, oxygen, secreted proteins, cytokines, inflammatory markers, fluidic pressure, substantially as shown in and/or described in connection with at least one of the figures, and as set forth more completely in the claims.

In accordance with an aspect of the present invention, there are provided methods for performing high-throughput cell, organ or multiple organs based assay in standard formats such as 6-well, 12-well, 24-well, 48-well, 96-well, 384-well or custom well plates.

In accordance with an aspect of the present invention, there are provided methods for performing high-throughput cell, organ or multiple organs based assay adapted to microfluidic chips with reservoirs in various array format.

In accordance with an aspect of the present invention, there are provided methods for organs such as brain, heart, lung, liver, gastrointestinal tract, skin, kidney, pancreas, bone marrow, skeletal muscles and other organs connected in series or parallel to each other.

In accordance with an aspect of the present invention, there are provided methods for screening drugs through aerosol nasal system to lung or oral drug to gut and study the toxicity of metabolites to other organs.

In accordance with an aspect of the present invention, there are provided methods to study the effect of the drug in blood circulation and pumping through the heart from other organs.

In accordance with an aspect of the present invention, there are provided methods to interface a disposable chip to a fluidic system to transport media or drug or nutrients with the cell/organ container to chip or well.

In accordance with an aspect of the present invention, there are provided methods to encapsulate and organize cells or organs within gel, extracellular matrix, filter, scaffold and/or reagents to grow cells or organs.

In accordance with an aspect of the present invention, there are provided methods to monitor the cells or organs and their interaction to drugs or electrical or mechanical stimuli using field potential, trans-epithelial electrical resistance, permeability, optical imaging, spectral measurements, gene expression or/and protein/cytokine/chemokine measurements.

In accordance with an aspect of the present invention, there are provided methods for recirculating fluids within a well or reactor through pumps, manifold fitted with O-rings to a microfluidic lid fitted with dispensers/suckers.

In accordance with an aspect of the present invention, there are provided methods to pump fluids from inner well to outer well or from outer well to inner well for different applications

In accordance with an aspect of the present invention, there are provided methods to perfuse fresh media to wells or reactors through push pumping system and waste/used media from wells or reactors through pull pumping system.

In accordance with an aspect of the present invention, there are provided methods to couple a recirculation system and a perfusion system as a single fluidic system using air pumps/valves or liquid pumps/valves or combinations. In accordance with an aspect of the present invention, there are provided methods to circulate fluids with a transwell or 3-d cell culture insert either from outside well to inside well or from inside well to outside well through filter or membrane or scaffold.

In accordance with an aspect of the present invention, there are provided methods to construct transwell with multiple cells or cell sheet combinations in layers or mixed in gel inside the inner well or combinations with outer well.

In accordance with an aspect of the present invention, there are provided methods to push and pull fluids equally from a well or reactor and maintaining the fluid level using a three way valve, vacuum or air pump to or from a reservoir by pushing or pulling the fluid.

In accordance with an aspect of the present invention, there are provided methods to pump the fluid in the forward direction and pump a small amount of fluid to the backward direction in order to hold the liquid level with air bubbles.

In accordance with an aspect of the present invention, there are provided methods to calibrate the height of the fluid with the flowrate of the fluidic pumping in and out of the reservoir by adjusting the number of strokes of pumping as a pulse width modulation.

In accordance with an aspect of the present invention, there are provided methods for pumping fluids from multiple wells or reactors using multiple set of pumping systems each providing one to one mapping.

In accordance with an aspect of the present invention, there are provided methods for fluidic control using multiple pumps from multiple reservoirs to wells as direct fluidic connections on a microfluidic plate with channels and droppers or pullers.

In accordance with an aspect of the present invention, there are provided methods for fluidic control using multiple pumps from multiple reservoirs to wells as binary divided fluidic connections on a microfluidic plate.

In accordance with an aspect of the present invention, there are provided methods for fluidic control using multiple pumps from multiple reservoirs to wells on different layers of a microfluidic plate.

In accordance with an aspect of the present invention, there are provided methods for fluidic control using multiple pump types such as dc pumps, peristaltic pumps, piezo electric pumps, electro-osmotic pumps or acoustic streaming pumps.

In accordance with another aspect of the present invention, there are provided methods for performing concentration gradient using splitting fluidic flow from two or more inputs of drug, growth factor, toxin, stimuli agents, other chemicals or reagents.

In accordance with yet another aspect of the present invention, there are provided methods for performing fluidic circulations within a well using a portable fluidic system.

In accordance with an aspect of the present invention, there are provided methods to connect recirculation system and perfusion system through a manifold and multilayer fluidic lid with two sets of fluidics.

In accordance with an aspect of the present invention, there are provided methods to interface reservoirs with fluidic lid using a user friendly manifold which mechanically provide an air or fluid tight seal with latch-closing top layer at an angle or straight.

In accordance with an aspect of the present invention, there are provided methods for manifold to make a tight connection through a tube adapter and O-ring on both sides of a fluidic connector array arranged in triangular or rectangular array.

In accordance with an aspect of the present invention, there are provided methods for connecting tubings from pumps to manifold through a side entry to avoid any movement of the tubings during operation.

In accordance with an aspect of the present invention, there are provided methods for manifold with circular or rectangular shallow pillars to press the O-ring area of the manifold with rapidly connecting microfluidic lid.

In accordance with an aspect of the present invention, there are provided methods for microfluidic lid with channels for dropping fluids arranged in a non-intersecting format on the top for perfusion fluidics and on the bottom for circulation fluidics.

In accordance with an aspect of the present invention, there are provided methods in the microfluidic lid for imaging the cells or organs through an open view area within fluidic dispensers' holes.

In accordance with an aspect of the present invention, there are provided methods of a narrower side in the microfluidic lid to interface with reservoirs/pumps through a fluidic O-ring array arranged in a 1-d or 2-d rectangular or triangular array.

In accordance with an aspect of the present invention, there are provided methods to guide tubings from the manifold through a side hole array so that the manifold can allow the microplate where the cells are to stay on the same level as the imaging plane on a microscope.

In accordance with an aspect of the present invention, there are provided methods in the manifold with groves for inserting lid and a shallow pillar locking mechanism to lock the lid for aligning the inlet/outlet ports.

In accordance with yet another aspect of the present invention, there are provided methods in the lid to pull fluid from the top well and dispense fluid in the bottom well using sucking fluidic tip and dispenser tip and vice versa.

In accordance with yet another aspect of the present invention, there are provided methods in the dispenser head with one or more dispensing ports arranged with multiple positions and pulling port arranged in an opposite end.

In accordance with yet another aspect of the present invention, there are provided methods to dispense fluids from one or more reservoirs and remove used fluid to a reservoir using a set of fluidic valves arranged outside the manifold through input/output ports.

In accordance with an aspect of the present invention, there are provided methods for microplate with alignment holes for inserting in to the manifold.

In accordance with yet another aspect of the present invention, there are provided methods for multiple fluidic devices ranging from single well system to multiwell and multilayer systems with additional features for electrical or optical monitoring and mechanical or electrical stimulation and drugs or chemicals screening.

In accordance with an aspect of the present invention, there are provided methods for connecting pumps at the microfluidic input/output ports in certain configurations so that the fluid with circulate between one or more wells.

In accordance with an aspect of the present invention, there are provided methods to measure the liquid level of each well using electrical impedance measurement using two gold coated or platinized electrode pins attached through holes in the lid so that corresponding pump/s causing fluid flow in to the well can be turned off or corresponding pump/s causing fluidic flow out of the well can be turned on to keep the fluid level constant.

In accordance with an aspect of the present invention, there are provided methods for impedance sensors for water level or trans-epithelial electrical resistance between inner well and outer wells using an array of electrodes attached to the fluidic lid.

In accordance with an aspect of the present invention, there are provided methods to measure water level based on impedance measurement circuits and feeding back through microcontrollers and electronic switches to control pumps.

In accordance with yet another aspect of the present invention, there are provided methods for performing fluidic circulations between two or more wells in series, parallel or combinations of series and parallel.

In accordance with yet another aspect of the present invention, there are provided methods for performing fluidic circulations between two or more wells in forward or backward directions.

In accordance with an aspect of the present invention, there are provided methods to hold a set of pumps and valves on a fluidic manifold so that the system will use no tubings

In accordance with an aspect of the present invention, there are provided methods perfusion from a fresh fluid reservoir in to a 6-well plate using a pair of fluidic pumps and a set of 12 fluidic valve.

In accordance with an aspect of the present invention, there are provided methods to perform simultaneous perfusion and re-circulations by a set of fluidic pumps and valves which can circulate through one way of the valves and perfuse through another way of the valves.

In accordance with yet another aspect of the present invention, there are provided methods to prepare a serial drug or reagents concentrations from a stock solution and a buffer using pulse fluidic mixing and dispensing through a set of valves in each well.

In accordance with yet another aspect of the present invention, there are provided methods for performing concentration gradient for drug or chemicals on same cells at various time intervals using two inlet and one outlet microfluidic setup.

In accordance with an aspect of the present invention, there are provided methods to heat the wells using a heater filament plate made of transparent electrode materials arranged in between the microplate and 6-well plate.

In accordance with an aspect of the present invention, there are provided methods to control CO2 and O2 ratio in the well plate by additional channels in the microplate for gas mixture to flow in to well plate.

In accordance with an aspect of the present invention, there are provided methods to hold microplate in the well plate tightly using gaskets so that hypoxia for the cells or organs can be controlled as well as imaging can be performed at the best magnification.

In accordance with an aspect of the present invention, there are provided methods to control perfusion in microfluidic chips with cells in gel or by themselves in reactors connected in series or well in channels.

In accordance with an aspect of the present invention, there are provided methods to perfuse media from reservoirs in to microfluidic channels holding cells or organs in gel as 3d or 2d culture.

In accordance with an aspect of the present invention, there are provided methods to perform perfusion of media in cells or organs in 3-d cell culture to form vascular network.

In accordance with an aspect of the present invention, there are provided methods to perfuse 2-D array of reactors in a standard well format using perfusion recirculation system in 2-D or 3d culture

In accordance with an aspect of the present invention, there are provided methods to detach array of electrodes to extract cells and to close tightly using silicone layer using wedges in silicone layer and/or manifold top metal layer

In accordance with yet another aspect of the present invention, there are provided methods to monitor drug concentrations and their interaction with cells or organs using impedance measurements and optical imaging on a manifold.

In accordance with yet another aspect of the present invention, there are provided methods to completely automate concentration gradient, washing, incubation, repeat iterative pulse fluidics and data/image acquisition.

In accordance with yet another aspect of the present invention, there are provided methods to prepare concentration profile using pulse width modulation of pumping of drug and buffer using precision of pumping flow rate and number of bits to form a pattern of binary codes for the pumps.

In accordance with yet another aspect of the present invention, there are provided methods to develop increasing or decreasing concentrations with alternate fluidic pulsing to produce homogeneously mixed concentrations.

In accordance with yet another aspect of the present invention, there are provided methods for microfluidic chips with one or more wells or reactors in series or parallel with one or more inputs and one or more outputs or one or more independent channels will one or more inputs and one or more outputs for cellular studies.

In accordance with yet another aspect of the present invention, there are provided methods to load fluids in a pumping system for perfusion or recirculation with independent inputs and output to proliferate, differentiate or vascular formation of cells or organs with fluidics.

In accordance with yet another aspect of the present invention, there are provided methods for microfluidic chips in 6, 12, 24, 48 or 96 well format or custom formats to grow cells or organs with automated fluidic perfusion or recirculation, imaging, cellular monitoring.

In accordance with yet another aspect of the present invention, there are provided methods for removable microfluidic chips to retrieve the cells after cellular in vitro assay to perform offsite measurements such as PCR or immunoassay.

In accordance with yet another aspect of the present invention, there are provided methods for microfluidic chips to rapidly connect to fluidic pumping system using a manifold and to measure optical or electrical parameters continuously.

In accordance with yet another aspect of the present invention, there are provided methods to hold reagents and battery with the system to operate remotely from an incubator with minimum controls on the system while fully controlled using a smart handheld device.

In accordance with an aspect of the present invention, there are provided methods to provide mechanical stimulation and/or electrical stimulation to heart or muscle or brain cells in a dog-bone like format within an insert.

In accordance with yet another aspect of the present invention, there are provided methods to perform mechanical and electrical stimulation along with fluidic perfusion using electromechanical actuators and electrical current/voltage connected through lid surface.

In accordance with an aspect of the present invention, there are provided methods to perform force measurements in functional muscle cells using XYZ stage and a force sensor

In accordance with yet another aspect of the present invention, there are provided methods to arrange multiple cells such as brain endothelial cells, Pericytes, astrocytes and neurons in scaffold or 3-D inserts and provide electrical activity from neuronal cells using microelectrode array.

In accordance with yet another aspect of the present invention, there are provided methods to culture endothelial cells on one side of the 3-D insert and Pericytes on another side together with astrocytes and neurons forming blood-brain-barrier.

In accordance with yet another aspect of the present invention, there are provided methods to develop microfluidic removable top and bottom fluidics using two sets of silicon layers and filter separating top and bottom fluidics.

In accordance with yet another aspect of the present invention, there are provided methods to form 2-D array of fluidic reactors one top and bottom layer separated by membranes with drug applications as a concentration gradient.

In accordance with yet another aspect of the present invention, there are provided methods to form vascularized cells in gel for different organs using series of expanding channels arranged in a serpentine format in elliptical or circular microfluidic inserts with perfusion along the sides of the main cell/gel channel.

In accordance with an aspect of the present invention, there are provided methods to perform 3-D cell culture with gel for a 2-D array of reactors in standard format and perfusion with finger channels

In accordance with yet another aspect of the present invention, there are provided methods to perform perfusion of such vascularized cells in gel using a separate set of channels with one or two media on either sides and with or without connecting their outlets.

In accordance with yet another aspect of the present invention, there are provided methods provided to load cells in gel on microfluidically connected reactors and to perform perfusion through a separate set of perfusion channels with fingers for stopping gel migration in to perfusion channel

In accordance with yet another aspect of the present invention, there are provided methods to form vascular cells in a 3-D printed scaffold that enable perfusion of 3-D tissues and to mechanically and electrically stimulate in addition to electrical monitoring using field potential signals.

In accordance with yet another aspect of the present invention, there are provided methods for developing a mechanical stretchable silicone chip with perfusion fluidics and electrical measurement using conductive polymer ink.

In accordance with yet another aspect of the present invention, there are provided methods for simultaneous electrical impedance and field potential measurements using interdigitated electrodes with point multielectrode array electrodes.

In accordance with yet another aspect of the present invention, there are provided methods conduction velocity measurements from electrogenic cells using 1-D electrode array with stimulation electrodes on one of the sides or at the center.

In accordance with yet another aspect of the present invention, there are provided methods for optical imaging of cells from the inner well using upright microscope with a Grin lens and from the outer well using an inverted microscope.

In accordance with yet another aspect of the present invention, there are provided methods to acquire field potential signals from cells on a 3-D insert using electrode sensors in the inner well and pads for spring loaded connectors in the outer top well separating bottom well.

In accordance with yet another aspect of the present invention, there are provided methods to measure field potential signals from top well of 3-D insert using spring loaded connectors resting on the top well using a circular printed circuit board equipped with viewing hole for imaging.

In accordance with yet another aspect of the present invention, there are provided methods to connect spring loaded connectors to top amplifier array connectivity circuit board forming an array for multiple wells.

In accordance with yet another aspect of the present invention, there are provided methods to perform simultaneous field potential measurement from 6-well plate with fluidic perfusion using top PCB with amplifier array and DAQ, 6-well plate electrodes sealed with bottomless 6-well plate and fixture to hold spring loaded connectors to connect the well electrodes.

In accordance with yet another aspect of the present invention, there are provided methods for perfusion fluidic inserts for multi-well plate with alignment holes, sucking tip hole and stands for adapting to standard well format.

In accordance with yet another aspect of the present invention, there are provided methods top—fluidic-pull insert with two set of holes for sucking from top well and dispensing securely to bottom well.

In accordance with yet another aspect of the present invention, there are provided methods for developing caps for fluidic reservoirs with inside and outside tube connectors and multiple screws liners for air-tight seal.

In accordance with yet another aspect of the present invention, there are provided methods for cascading multiple screw-caped reservoirs for easy handling so that the tubings are secured from twisting.

In accordance with yet another aspect of the present invention, there are provided methods to perform neurovascular drug screening for neurological disorders and monitor the cells using optical imaging, impedance and field potential signals.

In accordance with yet another aspect of the present invention, there are provided methods for 3-D cell culture using 3D printing of gel, scaffold and cells to perform fluidic perfusion and recirculation and to evaluate the cells using multiple modalities.

In accordance with yet another aspect of the present invention, there are provided methods to perform GPCR drug screening in 3-D cell or organ culture system and to perform pharmacokinetics or pharmacodynamics using multiple modalities.

In accordance with yet another aspect of the present invention, there are provided methods to perform fluidic perfusion, intra-well circulation and inter-wells circulation of organs over several weeks for pharmacological studies.

In accordance with yet another aspect of the present invention, there are provided methods to connect multiple monitoring sensors and activators with a Field programmable gated array or microcontroller and to communicate with different devices using Wi-Fi and BLE.

In accordance with yet another aspect of the present invention, there are provided methods to control DC pumps, peristaltic pumps in forward or reverse direction using MOSFET, optocoupler or DC-DC/LDO converters.

In accordance with yet another aspect of the present invention, there are provided methods to control the pumping system using a smart device application software.

In accordance with yet another aspect of the present invention, there are provided methods adapt the fluidic system in incubator, microscope and commercial imaging system and capable of fluidic operations.

In accordance with yet another aspect of the present invention, there are provided methods to heat the wells using microwave radio frequency or DC resistive currents to remove any condensed liquid on the 6-well plate surface that will object viewing of cellular images and/or to heat the media/wells to physiological temperature such as 37 deg C.

In accordance with yet another aspect of the present invention, there are provided methods to clean the lid and the pumping system using digestive enzyme cleaning solution from 6-wells with sufficient volume for cleaning.

In accordance with yet another aspect of the present invention, there are provided methods to automatically put together inserts using multiple layers alignment and pressing.

In accordance with yet another aspect of the present invention, there are provided methods to electroplate inserts with electrodes using a push-pull fluidics setup and spring loaded connectors arranged in layers of channel/well and gaskets.

In accordance with yet another aspect of the present invention, there are provided methods to manufacture tips for lids from conventional pipette tips by one or two ends cutting using layer or mechanical blades.

Further aspects, elements and details of the present invention are described in the detailed description and examples set forth here below.

### BRIEF DESCRIPTION OF THE DRAWINGS

The embodiments set forth in the drawings are illustrative and exemplary in nature and not intended to limit the subject mater degined by the claims. The following detailed description of the illustrative embodiments can be understood when read in conjunction with the following drawings, where like structure may be indicated with like reference numberals in which:

FIG. 1 shows a diagram of exemplary organs system with several organs connected to arteial and venous blood flow;

FIG. 2 shows a diagram of an exemplary organs on a chip system interacting from lung to other organs;

FIG. 3 shows a organ system connecting to and from heart;

FIG. 4 shows chip and system constituting the organ on a chip system;

FIG. 5 shows a device block diagram for organ on a chip system;

FIG. 6 shows block diagram for the recirculation and perfusion system;

FIG. 7A shows a diagram of exemplary design of fluidic pathway in 3-D cell culture system from bottom well to top well;

FIG. 7B shows a diagram of exemplary design of fluidic pathway in 3-D cell culture system from top well to bottom well;

FIG. 8 shows an example for 3-D cell culture system for organ on a chip with cell sheet and/or cells in gel/scaffold;

FIG. 9A shows schematics of push-pull system with pulling fluid from a well using a vacuum pump and pushing fluid to a well using an air pump and three way valves;

FIG. 9B shows voltage pulse diagram of synchronized valve and pump activation;

FIG. 10A shows schematics of push-pull system with pulling fluid from a well using a vacuum pump and pushing fluid to a well using an air pump and two way valves;

FIG. 10B shows voltage pulse diagram of synchronized valve and pump activation;

FIG. 11A shows schematics of push-pull system with bubble stopper by negative flow;

FIG. 11B shows voltage pulse diagram of synchronized valve and pump activation;

FIG. 12A shows a calibration graph for volume of fluid pumped or height of the liquid in the reservoir with the number of strokes required for pumping;

FIG. 12B shows a reservoir under pumping

FIG. 13 shows the pumping system consists of valves and pumps for each reservoirs connected in pushing or pulling fluids to or from wells respectively;

FIG. 14 shows schematic of reservoirs on one side and 6 well plate on the other side for user inputs, connected to a manifold for easy connect;

FIG. 15A shows the reservoirs organization and orientation for fluidic connection to manifold;

FIG. 15B shows the reservoirs organization and orientation for fluidic connection to valves and pumps;

FIG. 16 shows the recirculation system organized as an integrated system with the manifold;

FIG. 17 shows the combined recirculation and perfusion system organized as an integrated system with the manifold and lid feeding the 6-well plate;

FIG. 18A shows the Manifold for 6 Well system with latch closing at an angle;

FIG. 18B shows the Manifold for 6 Well system with latch closing parallel to the top closing piece;

FIG. 19A shows the Manifold for 6 Well system with pillars on the top piece to pressurize the lid to provide air tight connection;

FIG. 19B shows the Manifold with 12 holes for the placement of L adapters and tubings;

FIG. 19C shows the Manifold with wall for mounting pumps, valves and control system;

FIG. 19D shows the Manifold with extra pillars on both sides for alignment of the lid on the plate and grove for inserting the lid for aligning to the fluidic ports;

FIG. 20 shows the fluidic lid on a 6-well plate that can be connected to a manifold;

FIG. 21A shows fluidic lid bottom layer for fluidic transport from the well to manifold;

FIG. 21B shows fluidic lid channel layer for fluidic transport from the well to manifold;

FIG. 21C shows fluidic lid top cover layer with a provision to insert any sensors in to wells;

FIG. 21D shows insert in manifold with O-rings connecting to the manifold on one side and L adapters connecting to the pumps, valves or reservoirs;

FIG. 22A shows fluidic lid bottom layer with triangular fluidic ports configuration to the manifold;

FIG. 22B shows fluidic lid channel layer with triangular fluidic ports configuration to the manifold;

FIG. 22C shows fluidic lid top cover layer with triangular fluidic ports configuration to the manifold;

FIG. 22D shows insert in manifold with triangular fluidic ports configuration to the manifold;

FIG. 23A shows fluidic lid bottom layer for connecting pushing and pulling tips;

FIG. 23B shows fluidic lid channel layer for connecting pushing and pulling tips;

FIG. 23C shows fluidic lid top cover layer for connecting pushing and pulling tips;

FIG. 24 shows fluidic lid with provision to lock in to the manifold using alignment holes;

FIG. 25A shows fluidic dispensers with one or more holes to connect to lid to drop or pull fluids into or out of the wells

FIG. 25B shows fluidic dispensers with one or more open channels to connect to lid to drop or pull fluids into or out of the wells

FIG. 25C shows fluidic dispensers with one or more holes to push or pull fluids into or out of the wells

FIG. 26 shows an overview of products for cells, organs, human culture devices with multimodal activation and monitoring system

FIG. 27A shows lid that can be connected to the pumping system so that one well will feed in to another in a closed loop

FIG. 27B shows dispenser with holes and pipette tip

FIG. 28 shows lid that can be connected to impedance sensors for either water level management or transepithelial electrical resistance measurements

FIG. 29A shows electrical sensors on the top of the lid for impedance measurements;

FIG. 29B shows lid with dispensers connected to manifold on O-rings;

FIG. 30 shows simple impedance circuit as an input to water level management through a microcontroller;

FIG. 31A shows fluidic connection of all the six wells one feeding another as closed network;

FIG. 31B shows fluidic connection of three sets of two wells connect one to another and backwards;

FIG. 31C shows fluidic connection of two sets of three wells connect one to another as a loop;

FIG. 31D shows fluidic connection of four wells connecting one to another as a loop;

FIG. 31E shows fluidic connection of one well feeding back and forth with two other wells;

FIG. 31F shows fluidic connection of one well feeding back and forth with five other wells;

FIG. 32A shows fluidic connection of one reservoir feeding six wells and collecting waste back to another reservoirs using fluidic valves;

FIG. 32B shows fluidic manifold for FIG. 32A with 2 fluidic pumps and 12 fluidic valves for tubing free operation;

FIG. 32C shows fluidic connections for a single channel integrated recirculation and perfusion system;

FIG. 32D shows fluidic connections for a single channel integrated recirculation and perfusion system with used media filtering in to the fresh media in the same reservoir;

FIG. 32E shows fluidic connections for a single channel integrated recirculation and perfusion system adapted to 6-well format;

FIG. 33 shows fluidic connection of a reservoir drug reservoir mixing with a buffer reservoir to feed different drug concentrations to six wells and collecting waste back to another reservoirs using fluidic valves;

FIG. 34A shows lid integrated with serpentine or spiral electrodes made of transparent electrodes for microwave radio frequency/resistive heaters with pads for electrical connections;

FIG. 34B shows serpentine electrodes made of transparent electrodes for microwave radio frequency/resistive heaters;

FIG. 34C shows lid connected to extra heater plate with holes and electrodes for 6-well plates

FIG. 35 shows system connecting all the wells to carbon di oxide—oxygen mixtures for incubation;

FIG. 36A shows lid with separate channels and ports for connecting all the wells to carbon di oxide—oxygen mixtures for incubation;

FIG. 36B shows lid connected to 6-well plate through an air tight gasket for incubation;

FIG. 37A shows microfluidic chips with a series of reservoirs connected by channels to pump;

FIG. 37B shows microfluidic chips with a wells in channel connected to pump;

FIG. 38A shows a set of pumps connect to manifold to perfuse fluids from a set of reservoirs;

FIG. 38B shows a set of fluidic channels with reactors for cell culture or cellular assay to connect to manifold to perfuse fluids from a set of reservoirs;

FIG. 38C shows a set of fluidic channels with reactors for gel based 3-D cell culture or 3D cellular assay to connect to manifold to perfuse fluids from a set of reservoirs with closed gel channel;

FIG. 38D shows a set of fluidic channels with reactors for gel based 3-D cell culture or 3D cellular assay to connect to manifold to perfuse fluids from a set of reservoirs with closed gel channel;

FIG. 39 shows a set of fluidic channels with reactors for cell culture or cellular assay in standard 48 well format or custom format to connect to manifold to perfuse fluids from a set of reservoirs and releasing the waste to another reservoir;

FIG. 40A shows a chip design using multiple layers for extracting cells after culture and manifold design for leak proof connection;

FIG. 40B shows wedge structure to press for leak proof connection;

FIG. 41A shows a two inlet one outlet chip with multiple electrodes for drug screening

FIG. 41B shows Flashing and Flushing fluidic experiments with pulsed fluidics scheme for priming of both the fluids, washing with buffer and programing of nine concentrations with washing

FIG. 42 shows concentration patterns for generating different concentrations of drug for screening experiments to conduct on the chip

FIG. 43 shows user interface for concentration Gradient+Washing sets for drug toxicity screening

FIG. 44A shows manifold for drug screening with buffer and drug inlets and one outlet

FIG. 44B shows manifold for drug screening with O-rings for fluidic interface and slot for electrical connection

FIG. 45 shows manifold for electrical field potential measurements with spring loaded connectors

FIG. 46 shows cell culture system with buttons for user control, removable battery compartments and fluidic reservoir

FIG. 47A shows sketal muscle cell culture system with electrical and mechanical stimulation and perfusion

FIG. 47B shows 6 well plate sketal muscle cell culture system with electrical and mechanical stimulation and perfusion

FIG. 48A shows 6 well plate sketal muscle cell culture system with electrical pads for signals and voltages

FIG. 48B shows electromechanical stimulator and perfusion fluidics

FIG. 48C shows nano Newton force measurement on functional muscle cells under culture using XYZ stage.

FIG. 49 shows multiple cells in co-culture in top and bottom fluidic channel with electrical field potential monitoring electrodes and pads for blood brain barrier

FIG. 50A shows multiple cells in co-culture in top and bottom wells and insert

FIG. 50B shows coculture of cells in multiple pieces of fluidic devices for cell assay

FIG. 51A shows multiple pieces of fluidic devices for cell assay

FIG. 51B shows multiple layers of fluidic devices for the fabrication the cell-co-culture device for blood brain barrier

FIG. 52 shows blood brain barrier device in multi-well format with top and bottom fluidics

FIG. 53A shows microfluidic chips to study 3-D cell culture or vascularization of organs in gel with separate inlets connected to dispensers of the lid for media perfusion

FIG. 53B shows microfluidic chips that can be adapted to a well of 6-well plate for perfusion to study cell culture or vascularization of organs in gel

FIG. 53C shows microfluidic chips in 48-well format with cells in gel loaded in each reactors by flow

FIG. 53D shows each well is equipped with a perfusion channel in separate layer for media perfusion for vascularization of organs in gel

FIG. 54A shows microfluidic cell culture chip under mechanical stimulation, electrical stimulation, field potential measurements and drug screening for 3-D printed vascular tissues in gels or organs

FIG. 54B shows microfluidic chip fabrication with different layers

FIG. 55A shows microfluidic chip fabrication with channels, inlets, electrodes and waste

FIG. 55B shows cross sectional view of microfluidic chip with flexible electrodes and scaffold for 3D cell culture

FIG. 55B shows cross sectional view of microfluidic chip with flexible electrodes and scaffold for 3D cell culture

FIG. 55C shows cross sectional view of microfluidic chip with electrodes for 3D cell culture

FIG. 56 shows micro array electrodes for one directional field potential measurements and conductivity measurements

FIG. 57A shows compact optical imaging and measurements

FIG. 57B shows compact optical imaging from top well using GRIN lens and simultaneuous bottom well imaging

FIG. 58A shows 3D inserts with small well on the top electrodes in the middle layer and potential cell culture on the bottom well for insert

FIG. 58B shows 3D inserts with hole to bottom well perfusion

FIG. 58C shows 3D inserts with fabricated electrodes

FIG. 59A shows measurement electrical fixture for 3D inserts connecting to recording circuit board

FIG. 59B shows measurement electrical fixture for 3D inserts using spring loaded connectors

FIG. 60A shows 96-well based field potential measurement system

FIG. 60B shows 6-well based field potential measurement system for 3D inserts

FIG. 60C shows 6-well based field potential measurement system with bottom electrodes

FIG. 60D shows side view 6-well based field potential measurement system

FIG. 61A shows a 3D insert with holes for fluidic transport from bottom well and alignment

FIG. 61B shows a 3D insert with fluidic transport directions and crosssection of multiple layers

FIG. 61C shows a 3D insert construction with multiple layers

FIG. 62 shows a 3D insert construction with multiple layers for top pull and bottom drop dispensers

FIG. 63A shows crosssectional cutout view of screw cap for reservoirs

FIG. 63B shows screw cap for reservoirs showing multiple screw thread for airtight sealing

FIG. 64 shows array of screw cap reservoirs connected in series or parallel blocks

FIG. 65 shows instrumentation for impedance and field potential measurements and pump control for biochip

FIG. 66 shows instrumentation for multiple sensor measurements, imaging, battery monitoring and pump control for biochip

FIG. 67 shows instrumentation for multiple pumps forward or reverse flow control for biochip

FIG. 68A shows smart device application software (App) for single pump control

FIG. 68B shows smart device application software (App) for 6-well control system

FIG. 69A shows block diagram for neurovascular assay with multiple modalities

FIG. 69B block diagram for instrumentation and monitoring cell/organs using multiple modalities

FIG. 70A shows block diagram for muscle cell/organ assay with multiple modalities

FIG. 70B shows block diagram for cardiovascular cell/organ assay with multiple modalities

FIG. 71A shows block diagram of drug screening with GPCR drugs using cell/organ assay with multiple modalities

FIG. 71B shows mechanism of GPCR drug on biomarkers for Alzheimer's disease

FIG. 72 shows fluidic operation for organ interaction system assay

FIG. 73A shows adaptation of the fluidic system in a commercial imaging system

FIG. 73B shows adaptation of the fluidic system in a commercial incubator system

FIG. 73C shows adaptation of the fluidic system in a commercial microscope system

FIG. 74A shows cleaning setup for cleaning pumps and lid using digestive enzyme cleaning solution

FIG. 74B shows side view of cleaning setup for cleaning pumps and lid

FIG. 75 shows automated manufacturing setup for aligning and layering of lid and insert products

FIG. 76A shows electroplating setup for sensing chip using flow of electroplaing solution

FIG. 76B shows side view of electroplating setup using push pull fluidic flow

FIG. 77A shows batch preparation of tips from pipette tips using mechanical cutter or laser

FIG. 77B shows batch preparation of tips with customized height and diameter for lids

### DETAILED DESCRIPTION

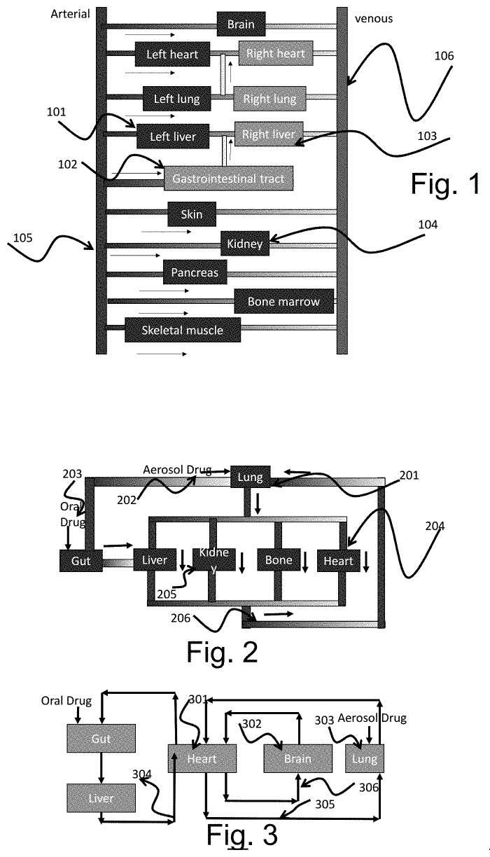

The following description contains specific information pertaining to implementations in the present application. The drawings in the present application and their accompanying detailed description are directed to merely exemplary implementations. Unless noted otherwise, like or corresponding elements among the figures may be indicated by like or corresponding reference numerals. Moreover, the drawings and illustrations in the present application are generally not to scale, and are not intended to correspond to actual relative dimensions. The focus of the invension is to develop a human system for drug screening using cellular and organ models as shown in FIG. 1. Different individual organs are connected in series 101, 102 or parallel 103, 194 to arterial 105 and venous 106 blood for the model of human system so that drug screening, disease modeling and several research acitivities can be carried out. The drugs can enter human body throgh lung 201 in the case of aerosol drug 202 and through gut in case of oral drug 203 as in FIG. 2. Several organs such as heart 204 and kidney 205 are connected parallel and circulated 206 with lung. As heart 301 is a central blood system model of human can be performed by connecting several organ system such as brain 302, liver 303 and lung 304 in parallel circulating 305 to heart as in FIG. 3.

Design and Development of Recirculation and Perfusion Fluidic System

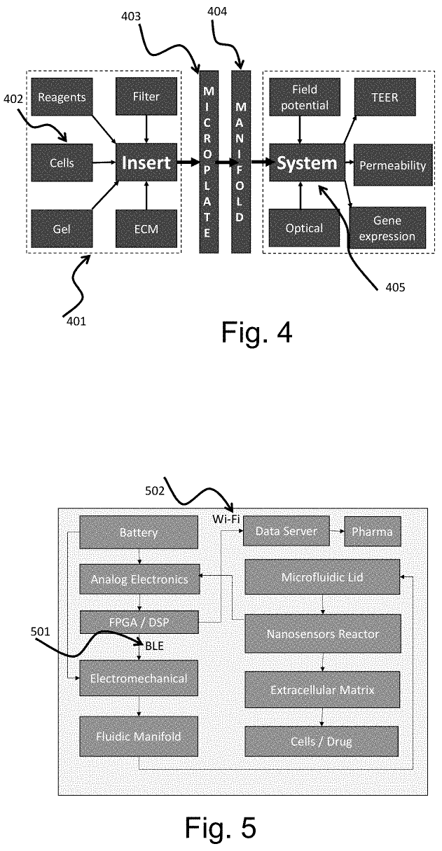

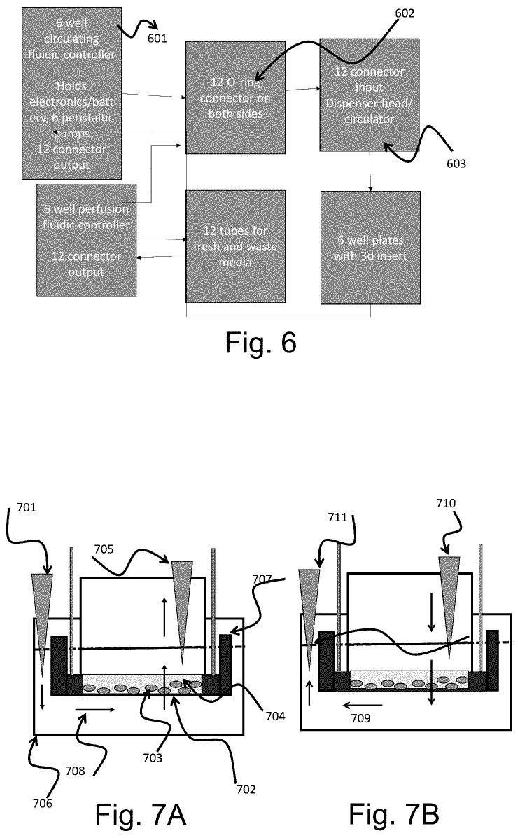

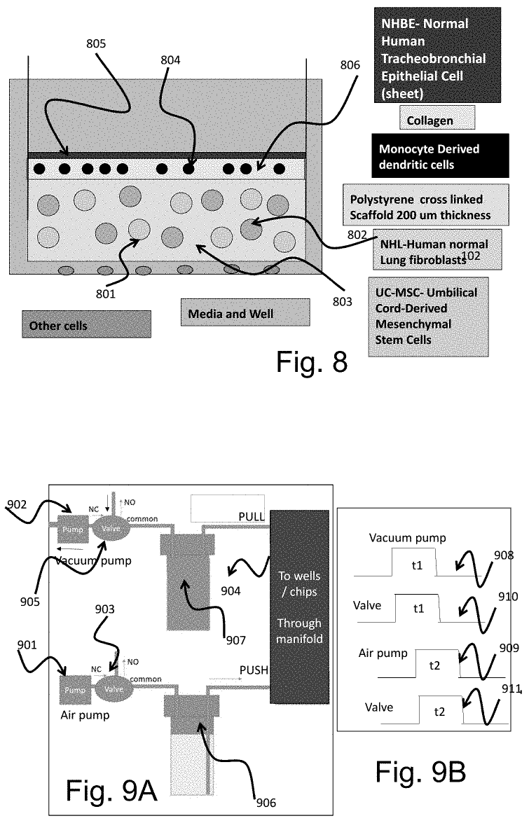

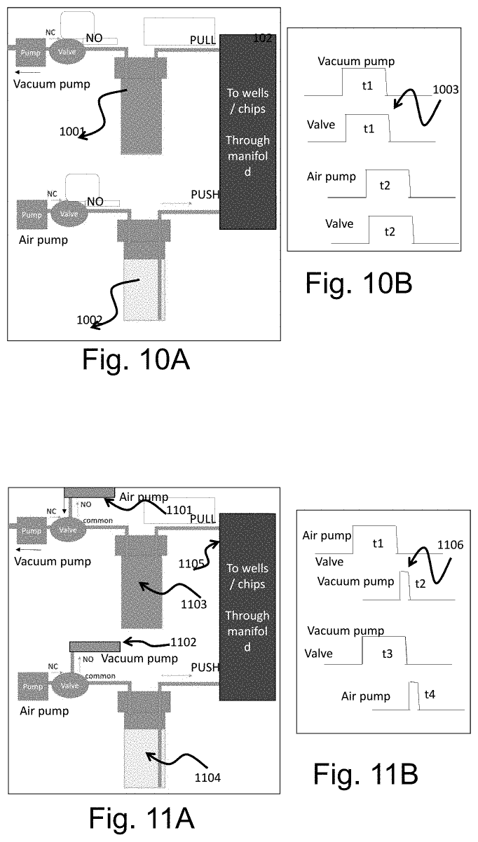

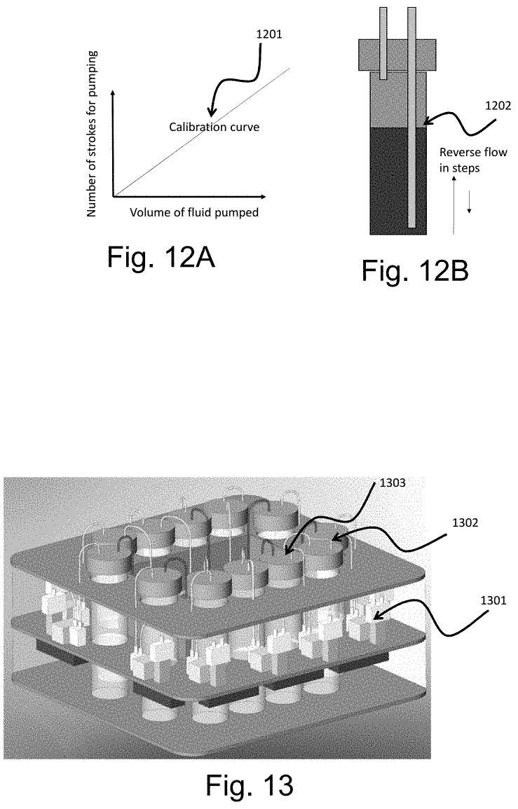

The system consists of a microfluidic chip, microplate, manifold and control/measurement system as in FIG. 4. In the microfluidic chip 401, cells or organs 402 by themselves or in gel or scaffold or filters with reagents and extracellular matrices are cultured. Microplate 403 transport fluids from reservoirs through the manifold 404 to the cells or organs. The system 405 can be used for drug screening using optical or electrical based biomarkers from the cells or organs and the data will be transmitted through electronics to a data server for pharmaceutical analysis as in FIG. 5. Bluetooth low energy (BLE) communication is used for controlling the instrumentations and Wi-Fi is used for data or image acquisition from the system. The fluidics in 6 well plate system 601 flow from fresh media tube or flask through pumps or valves through manifold with O-ring connector 602 to microplate with dispenser head 603 to wells as in FIG. 6. There are two directions of flow within the cell culture with inserts are possible as in FIG. 7A and FIG. 7B. The fluid can enter through the dispensing pipette 701, through filter 702 cells 703 and scaffold 704 and exit through the pulling pipette 705. The wells 706 of the six well plate can hold the inserts 707. The fluidic system can be configured in both ways 708, 709. The fluid can enter through the pipette 710 in the inner well and exit throug the pipette at the outer well 711. An example of 3D cell culture with cells 801, 802 in gel and scaffold 803 for 3-D cell culture with multiple cells 804, 805 in collagen 806 on transwell insert is presented in FIG. 8. In order to transport fluids from a reservoir 906, a push 901 and pull 902 technical can be used as illustrated in FIG. 9A. In the push subsystem an air pump pushes air through a valve 903 in to the reservoir to the well 904 while in the pull subsystem the fluid from the well is pulled in to a reservior 907 using a vacuum pump through another valve 905. The three way valves 903, 905 helps in venting the fluid in the reservoir between any pumping. The time pulses of pump 908, 909 synchronized with valve 909, 910 is presented in FIG. 9B. In FIG. 10A, the resevoirs 1001, 1002 are not vented so that the pressure builds up. The corresponding time pulse 1003 is shown in FIG. 10B. In another case, shown in FIG. 11A, additional pumps 1101, 1102 to provide a pulse flow in the opposite direction are used. This will help to avoid any drift in the level of the fluid from the reservoirs 1103, 1104 or well 1105. The corresponding fluidic pulses 1106 are presented in FIG. 11B. A calibration curve 1201 is developed as in FIG. 12A using the volume of fluid pumped to the number of strokes for pumping successive flow. In order to account for the decreasing height as in FIG. 12B the calibration curve is used for pumping trajectory 1202 from the reservoir. In FIG. 13, a 6-well based pumping system with push pull technique with two pumps and valve 1301 is shown. This system pumps fresh media from a reservoir 1302 and pumps back used media to another reservoir 1303 individually from all 6 wells.

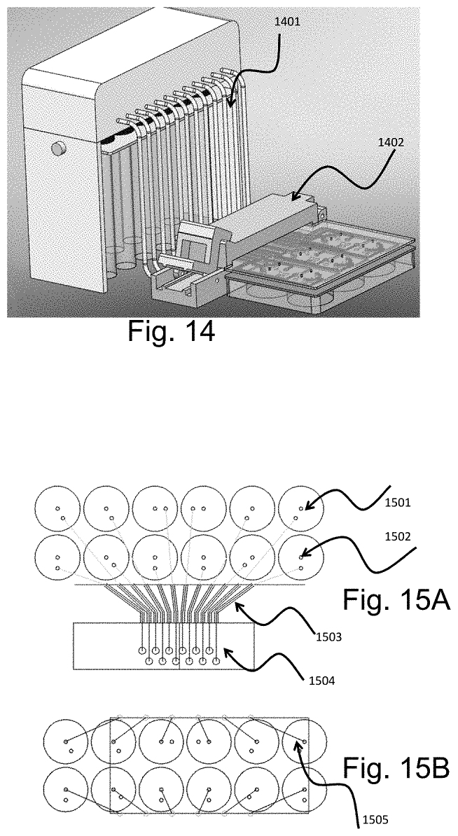

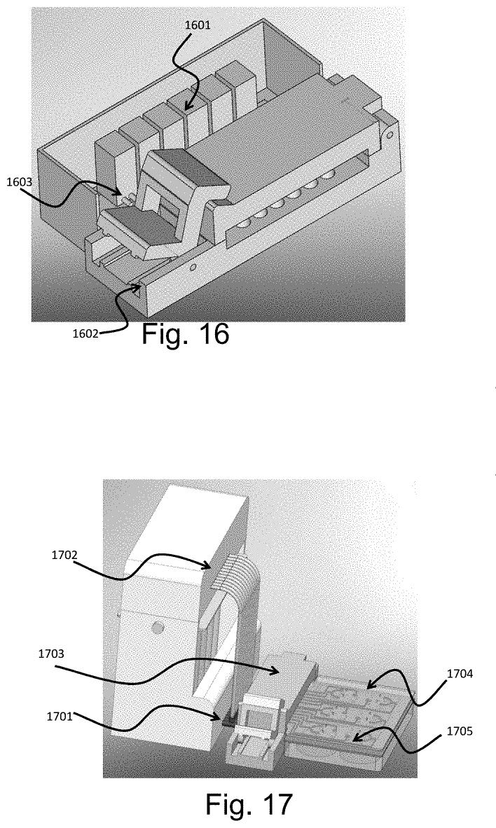

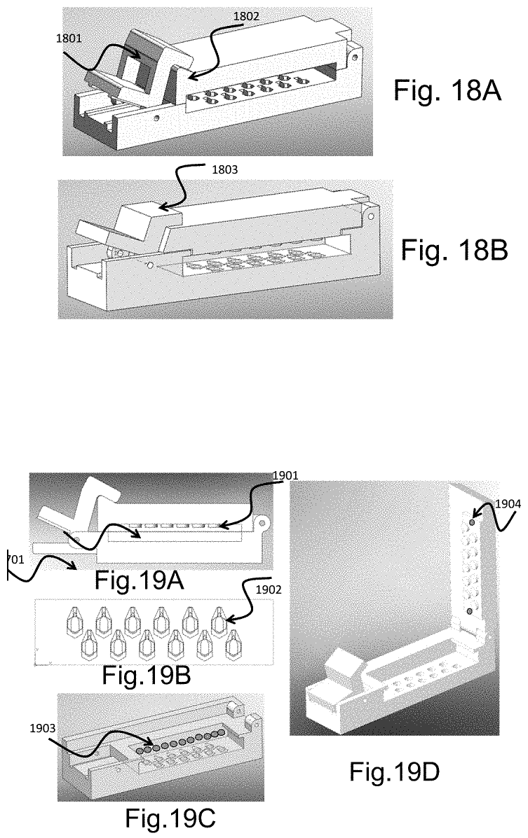

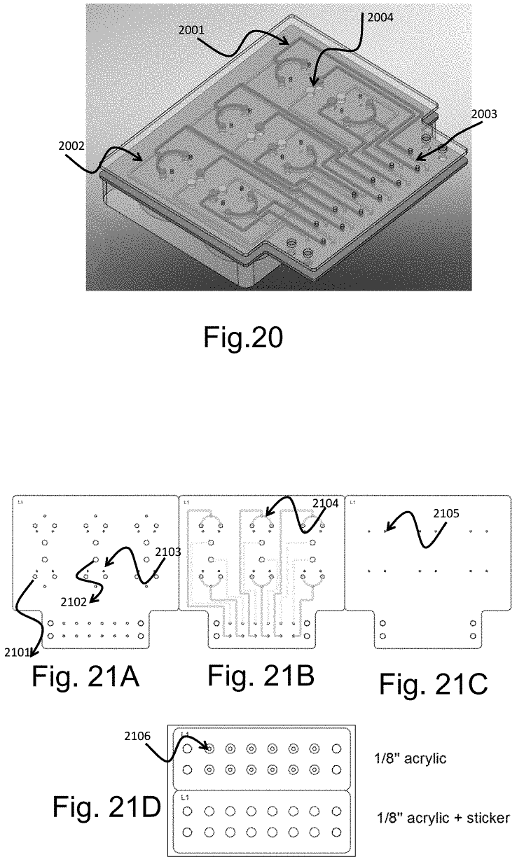

FIG. 14 shows the complete fluidic system which pump fluids from reservoirs through the manifold and microfluidic plate in to 6-well plate. This system can be extended to 12, 24, 48, 96 or any custom wells. The positions of the reservoirs with two holes 1501,1502 for fluid incoming and outgoing and tubings 1503 to manifold 1504 are designed to flow constant fluidic flow rate with out any disturbances as in FIG. 15A. In FIG. 15B, tubings 1505 to valves and pumps are designed. The recirculation system for 6 well plate consists of 6 pumps 1601 which can be peristalic or membrane pumps or PZT pumps and are connected to a manifold 1602 with straight tubing as in FIG. 16. FIG. 17 shows a fluidic system with recirculation 1701 and perfusion 1702 control connected to a manifold 1703. The microplate consists of two sets of fluidics in the top 1704 and bottom 1705 layer so that circulation and perfusion can be carried out in parallel or series within a well or inter-wells. The manifold in FIG. 18A with angular latch 1801 and angular top piece 1802 provides high force to close the manifold for air tight operation. The manifold with straight top piece and straight latch 1803 as in FIG. 18B will provide sufficient force to press the microplate for low pressure applications. The top piece of the manifold will have circular or rectangular pillars 1901 to press the microplate within the manifold as in FIG. 19A. The bottom piece of the manifold will have groves 1902 to hold a L-adapter as in FIG. 19B. The tubes from L-adapters are connected to the pumps through a side holes 1903 for all the 12 L-adapters as in FIG. 19C. The manifold will also have extra alignment pillars 1904 as shown in FIG. 19D to align microplate so that microplate can be inserted in the manifold blindly. In FIG. 20 a microplate with top 2001 and bottom 2002 fluidic channels are shown which starts or ends at the manifold ports 2003 and wells 2004.

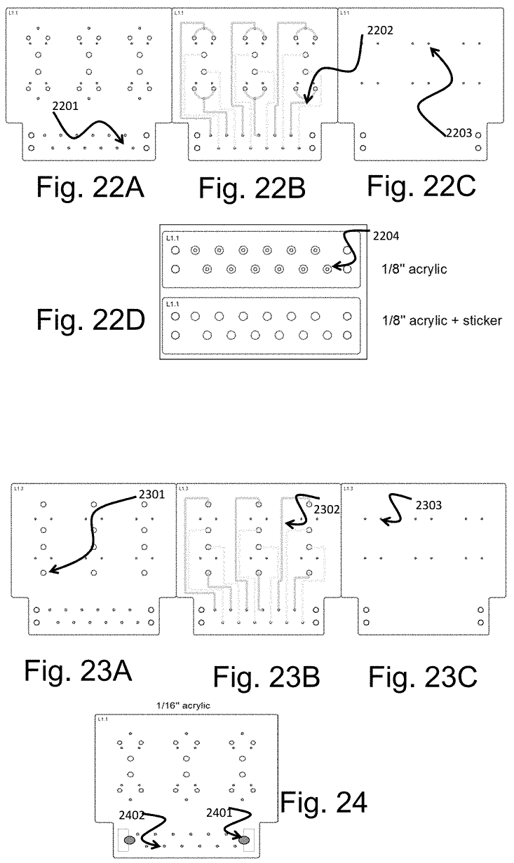

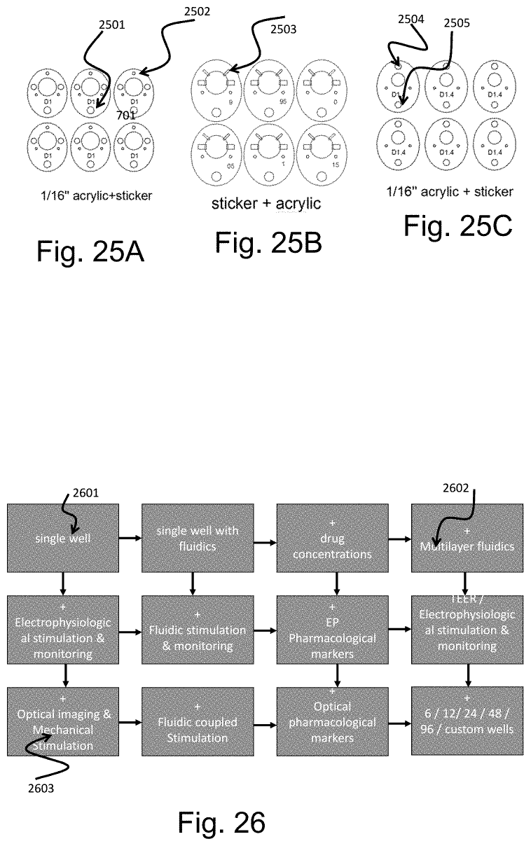

The microplate can be fabricated in three layers. The bottom layer will have holes for dispensors 2101, puller 2102 and any sensors 2103 as in FIG. 21A. The middle layer as shown in FIG. 21B will have holes for dispensor, pullers and also channels 2104 for the fluidics. The top layer as shown in FIG. 21C will have holes 2105 for sensor probes such as gold or platinum coated pins for water level sensing or impedance measurement. Two plates shown in FIG. 21D are inserted in the manifold to interface the microplate with fluid tight O-rings 2106 and accommodate L-adapter for connecting to tubings. The side entry of the tubings at the manifold is important so that the top piece can be free of any tubings. Any tubings at the top piece will affect the performance of the fluidic system. Therefore, in order to organize the tubings as side entry, triangular lattice for fluidic ports at the manifold and so microplates are considered. The bottom layer of such microplate with triangular lattice ports 2201 will have holes for dispensors, puller and any sensors as in FIG. 22A. The middle layer as shown in FIG. 22B will have holes for dispensor, pullers and also channels 2202 for the fluidics. The top layer, as shown in FIG. 22C will have holes for sensor probes 2203 such as gold or platinum coated pins for water level sensing or impedance measurement. Two plates shown in FIG. 22D are inserted in the manifold to interface the microplate with fluid tight O-rings 2204 and accommodate L-adapter for connecting to tubings. In the case of fluidic system that pulls fluid from the top well and drops the fluid in to the bottom well, the fluidic dispenser and puller has only one hole 2301 to provide more space for cell or organ imaging. The bottom layer of such microplate will have one hole for dispensors and one hole for puller and additional hole for any sensors as in FIG. 23A. The middle layer as shown in FIG. 23B will have holes for dispensor, pullers and also channels 2302 for the fluidics. The top layer, as shown in FIG. 23C will have holes 2303 for sensor probes such as gold or platinum coated pins for water level sensing or impedance measurement. The microplate also has additional alignment holes 2401 near the ports 2402 where it will be inserted in to the manifold for alignment as in FIG. 24. The microplate will hold dispenser and puller at the space above the wells. FIG. 25A shows a dispensor head with one hole 2501 for pippette top insertion and three holes 2502 for droping fluid in to the well. It can also drop fluid in to the top well in case of 3-D plate with inserts. FIG. 25B has channels 2503 at the dispensor or dropper so that the fluid will be dispensed for better mixing. FIG. 25C shows the dispenser head for attaching two pipette tips 2504,2505 which can be two different heights so that fluid can be pulled or dispensed from or to the top or bottom wells.

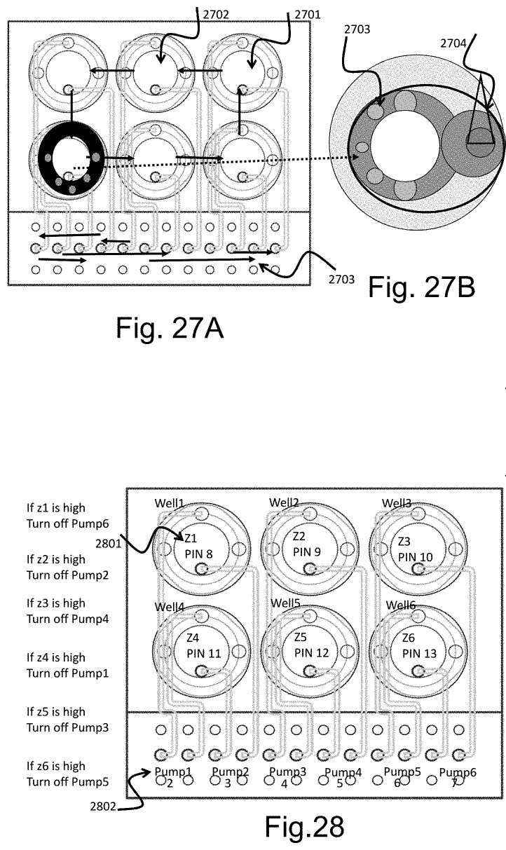

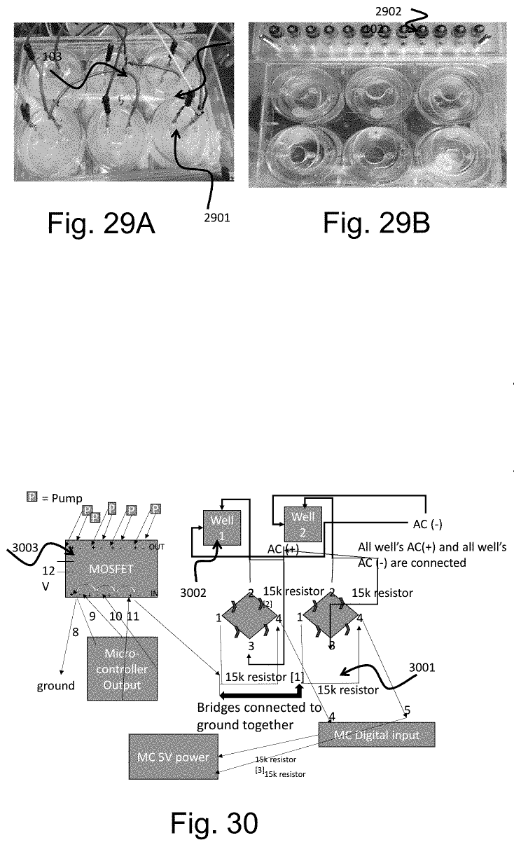

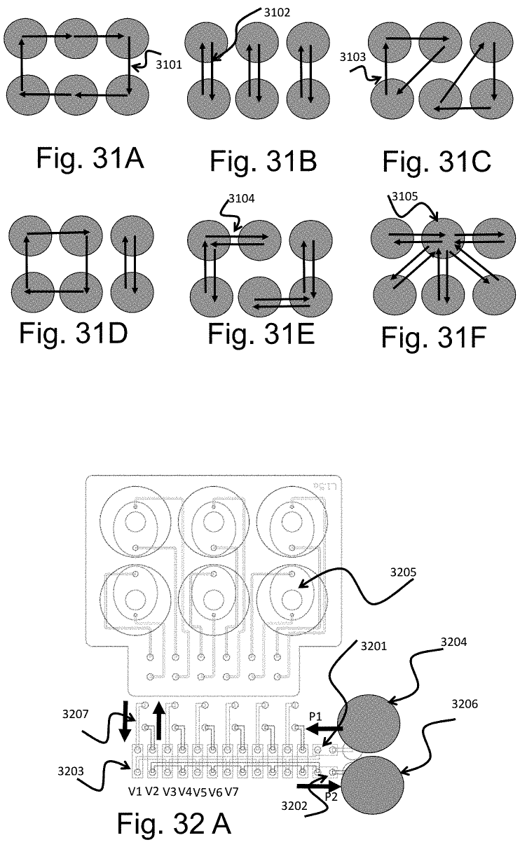

FIG. 26 shows the overview of multiple systems with increasing features and complexity from simple single well 2601 to multilayer fluids in multi-well format 2602 and capable of providing stimulation and monitoring of multimodal parameters 2603. FIG. 27A shows flow of fluid from one well 2701 to another well 2702 in 6-well format. This will help in interacting organs from one well to another well. Such fluidics are configured by connecting inport and outport pumps in a specific order 2703 required for interacting the organs. FIG. 27B is a view of fluid dropper in the well with dropping holes 2704 and pipette tip 2705 fluid puller. In order to monitor the levels of fluids in all the wells, impedance 2801 based monitoring is employed as in FIG. 28. The impedance sensors will detect if they are touching the fluids. If the impedance of a particular well sensors is low beyond a threshold, the pumps 2802 responsible for pumping in to the well is turned on as in FIG. 28. FIG. 29A shows the location of pins 2901 in the well. A printed circuit board with view holes for optical imaging of cells/organs is used to attach the sensor pins and connect to electrical circuits for impedance measurements. FIG. 29B shows the microplate with dispensors and fluidic ports 2902. FIG. 30 shows simple impedance measurement 3001 and control of pumps 3002 through switching circuit for two of the wells 3003. The microplate can be configured with channels and fluidic connections at the ports to perform several models for organ or human systems. FIG. 31A shows the circulation 3101 of fluids across all the 6 wells. FIG. 31B shows three sets of circulation of fluids between two wells 3102. FIG. 31C shows two sets of recirculation across three wells 3103. FIG. 31D shows recirculation across 4 and 2 wells. FIG. 31E shows recirculation of fluids from or to a particular well to two other wells 3104. In FIG. 31F, fluidic recirculation is configured from one well 3105 to all the other wells and back.

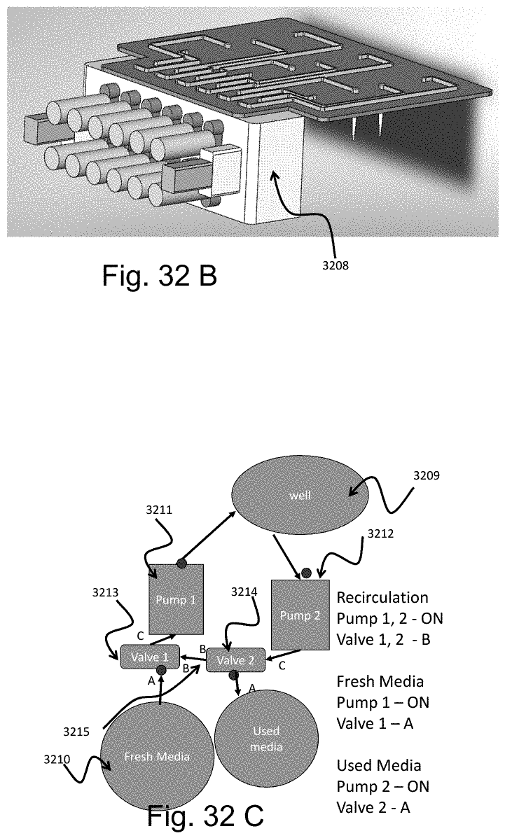

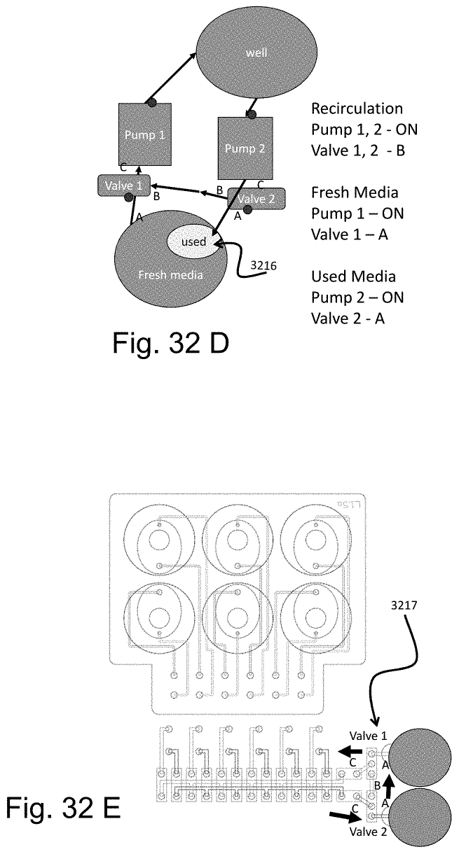

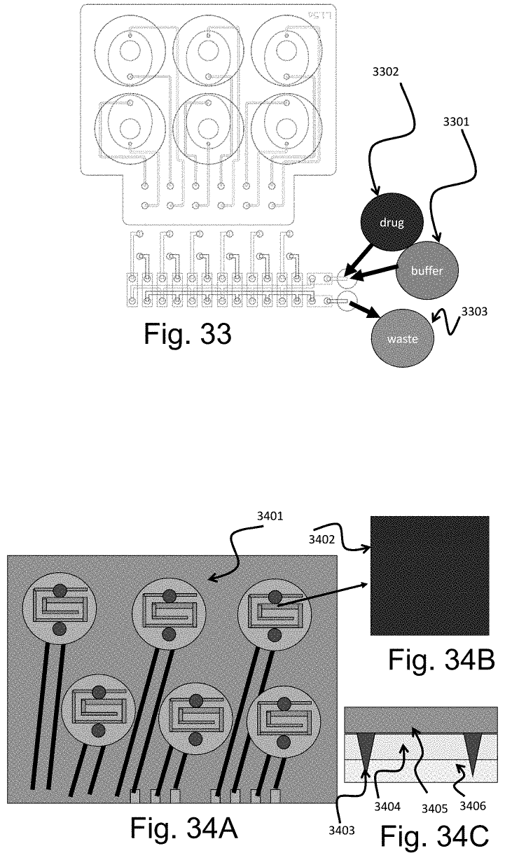

In another design, the perfusion of fluids can be performed using a set of liquid pumps and valves which are compatible with all the liquids such as cell media, buffer, drug, solvents. FIG. 32A shows a design with two pumps 3201, 3202 and 12 set of valves (v1, v2, v3 . . . v12) 3203. From the fresh media reservoir 3204 pump P1 3201 pumps in to the wells 3205 through one of the 6 valves (v1, v3, v5, v7, v9,v11) 3203 connected together with the fresh media reservoir. The used media from the wells 3205 are pumped by P2 3202 through a set of 6 valves (v2, v4, v6, v8, v10, v12) connected together on one side in to another reservoir 3206. All the pumps and valves are connected to a manifold and any tubings can be avoid using channels 3207 in the manifold 3208 shown in FIG. 32B. The control system is designed under the manifold to develop a compact system. FIG. 32C shows a fluidic system that can perform recirculation within a well 3209 and perfusion of new media 3210 in to the well using two pumps 3211, 3212 and two three way valves 3213, 3214. In order to perform recirculation, both the pumps are turned ON and the valves are switched to B positions 3215. In order to pump the media into or out of the well the corresponding pump is turn on while the valves are switched to A positions. In FIG. 32D, the used media is purified by a filter 3216 so that the used media can be pumped in to the top of the filter and the fresh media is pumped from the bottom. Any toxin or harmful substance of high size is filtered. In FIG. 32E, the single well system shown in FIG. 32D is applied to FIG. 32A so that 6 well recirculation and perfusion 3217 can be perform by programing the valves and pumps. Preparation of drug concentrations are performed using three reservoirs such as buffer 3301, drug 3302 and waste media 3303 as in FIG. 33. This setup can be used for perfusion with a concentration of drug through out the assay process.

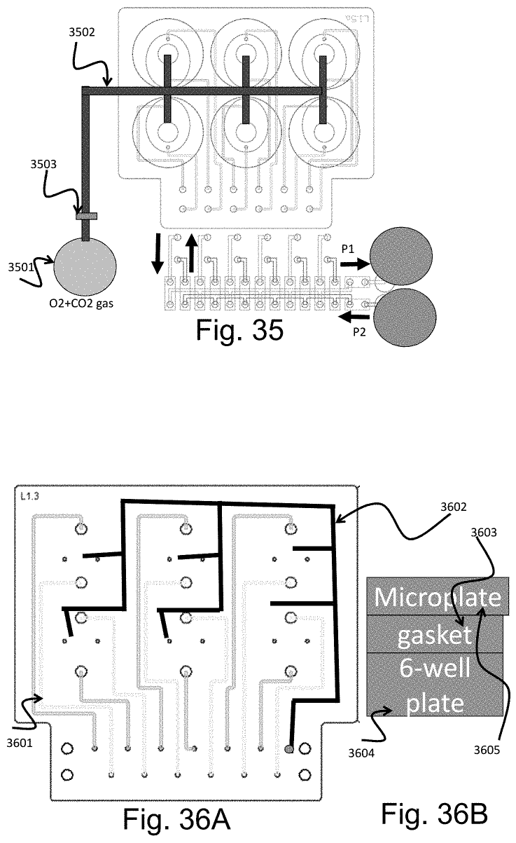

A heater plate with transparent heating filaments with holes for fluidic tips is developed as in FIG. 34A for heating the cell or organ culture while imaging. This will avoid any condensation of media on the plate that will interfere with the imaging. FIG. 34B shows the serpentine or double spiral heating filaments 3402 fabricated on a glass plate. FIG. 34C shows the side view of microplate with pipette tips 3403, heater plate 3404 inserted between the microplate 3405 and 6-well plate 3406. In FIG. 35, mixed CO2+O2 gas 3501 is passed on to the cells or organs through fluidic channels 3502 in the microplate from a CO2 cyliner with filter 3503. FIG. 36A shows the microplate fabricated with liquid channels 3601 and gas channels 3602 for media exchange. A gasket shown in FIG. 36B is connected between the microplate and 6-well plate to avoid any wastage of gases. Since this system will serve as a hypoxia chamber to adjust oxygen concentration from 100% to 0%. Programming oxygen and carbon-di oxide gas ratio can be achieved by a pair of gas valves. One of the significant of this hypoxia chamber is that there is no plastic or any other sheet in between the microscope imaging under the well plate so that imaging can be carried out at the maximum magnification. The hypoxia chamber is baseless and a gas gasket 3603 will fit the well plate 3604 with the microplate 3605 gas tight.

Development and Fabrication of Chips for Multimodal Monitoring

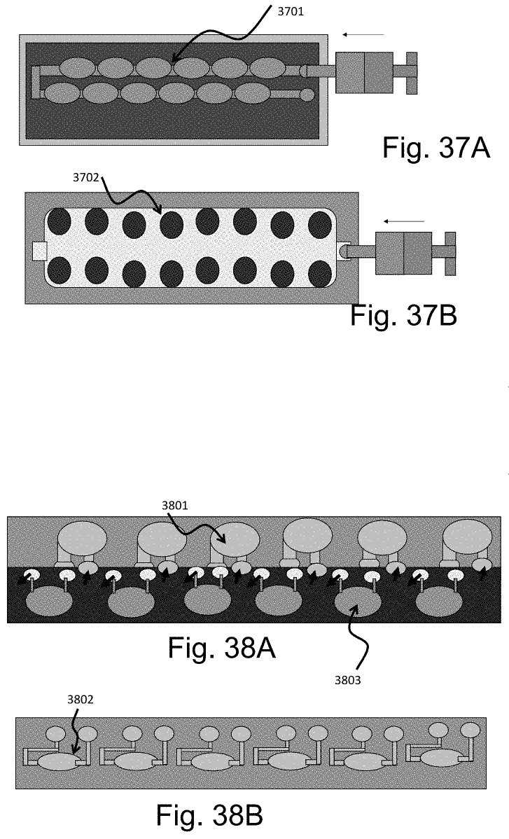

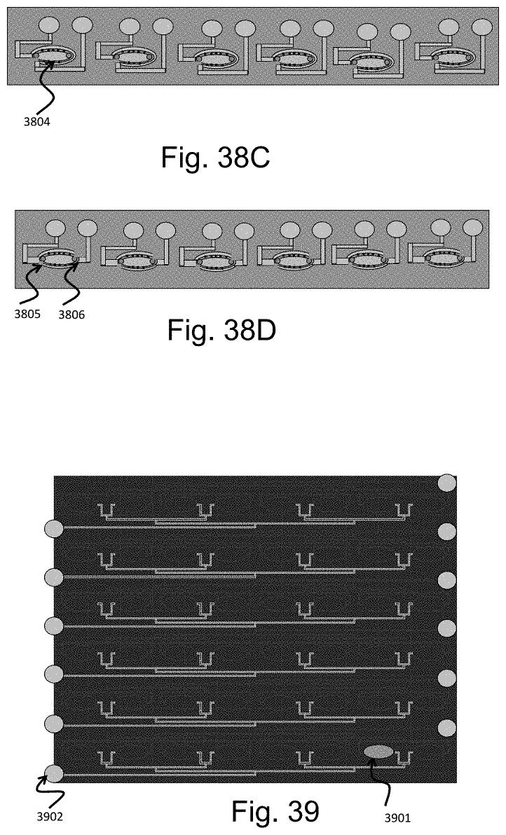

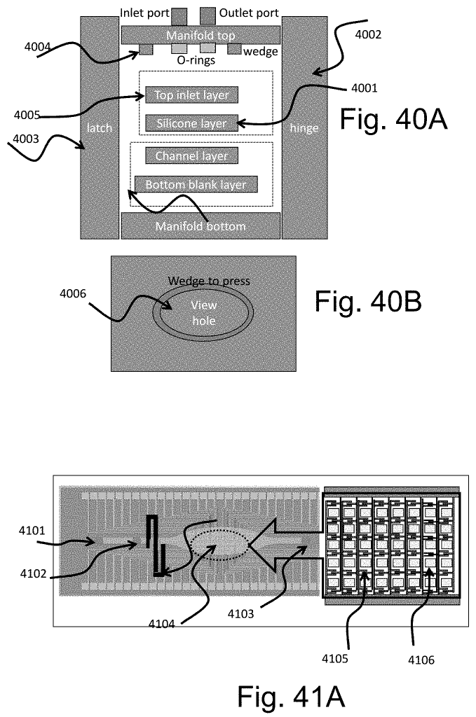

Microfluidic chips can be interfaced with the recirculation or pumping system. FIG. 37A shows a simple microfluidic chip with 12 reactors 3701 which can be automatically perfused or recirculated with media. FIG. 37B shows 16 wells 3702 in a channel for cell or organ culture or 3-d culture with gel. In FIG. 38A, the pumping system with manifold containing 6 reservoirs 3801 is shown. Microfluidic reactors 3802 as in FIG. 38B can be connected in series with the pumps 3803 so that media can be exchanged from reservoirs 3801. These reactors can be used for 3D cell or organ culture as in FIG. 38C where media can be perfused through connected finger channels 3804. The perfusion can also be performed from one side 3805 of the ellipsoidal reactor to the other side 3806 as in FIG. 38D. The reactors can be arranged in multiple well format as shown in FIG. 39. In this chip cells are loaded in the reactors 3901 and perfusion is performed through side ports 3902. The top layer can be removed to retrieve the cells. In these chips a silicone layer 4001 is used for locking the reactors fluid tight using a latch 4003 and hinge 4002 arrangement as in FIG. 40A. A wedge 4004 created on the manifold top piece 4005 will press the reactors for fluid tight operations and the manifold will have view holes 4006 for optical imaging as shown in FIG. 40B.

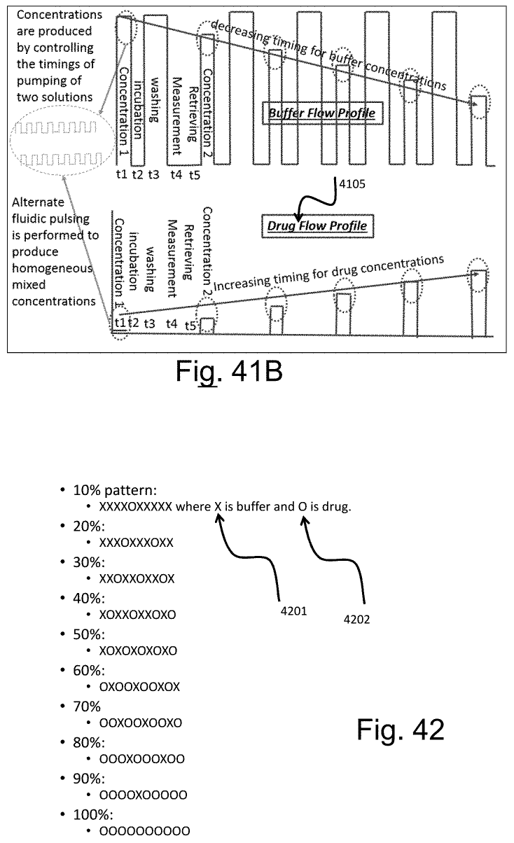

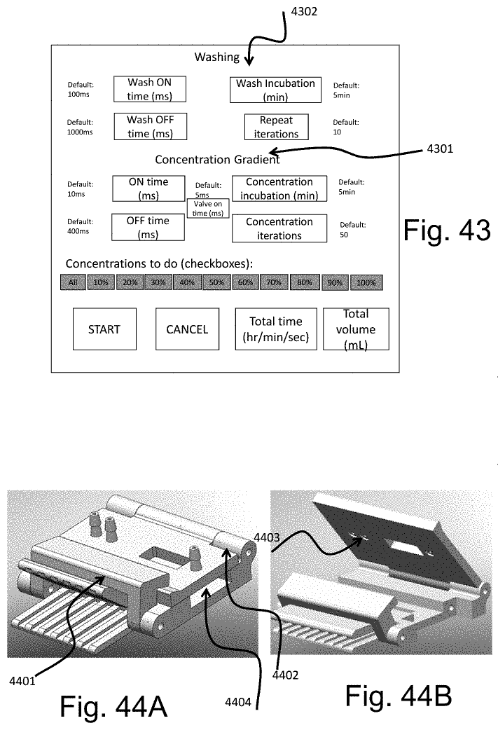

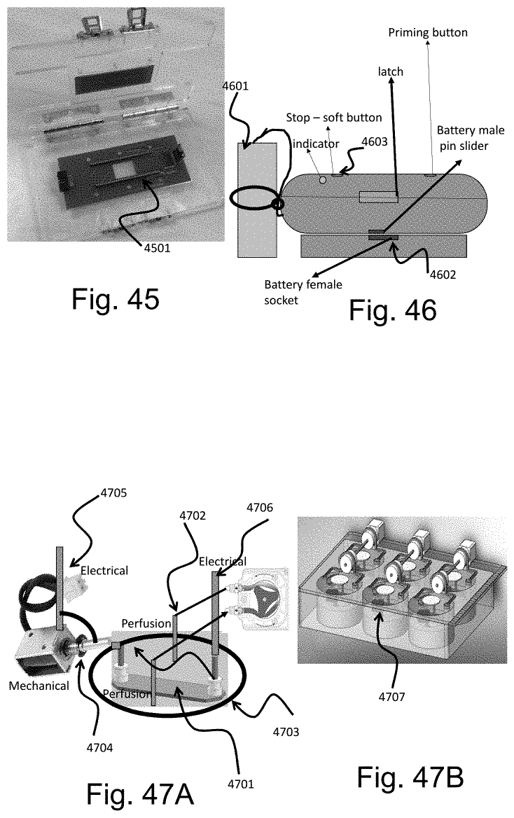

FIG. 41A shows a drug toxicity screening chip. The chip will consist of two or more inlets 4101,4102 for drug, cell media/wash buffer and one outlet 4103 for waste. The cell culture chamber/reactor will contain 64 electrodes 4104. Interdigitated electrodes to measure impedance and electrode pads to measure field potential signals are fabricated on the same surface where the cells are seeded. The chip operates leak-proof and bubble-free for priming, washing step and drug concentration generation. The cells are delivered manually at the reactor and perfusion of media/reagents is carried out by electronic control after locking in the manifold. Fluidic pulses are generated according to FIG. 41B for drug concentrations for screening. The patterns on buffer 4201 and drug 4202 in order to produce a particular percentage concentration is shown in FIG. 42. We will develop a portable system with temperature/humidity tolerance instrumentation that can operate remotely in a lab incubator. A software for generating concentrations 4301 of one fluid over other fluid was developed and the established a fluidic scheme for serial drug concentration using train of fluidic pulses was tested as shown in FIG. 43. Exposing drug concentrations one by one on the cell in culture is flashing 4301 and washing each concentration before flashing another concentration is flushing 4302. A typical ‘Flashing and Flushing’ fluidic experiment will run for 2 -10 hrs to perform 5-10 concentrations sandwiched by incubation, washing, measurement, retrieving steps. In FIG. 44A and FIG. 44B the manifold with latch 4401 and hinge 4402 used for the drug screening chip is shown. The manifold will have O-rings 4403 at the input/output ports for leak-proof interface of the chip. The manifold will also have provision for electrical edge connector 4404 for impedance measurements. FIG. 45 shows the fixture for field potential measurement using spring loaded connectors 4501. The system shown in FIG. 46 will have reservoirs for cell media/buffer and drugs. It will also have battery backup 4602 and buttons for operation.

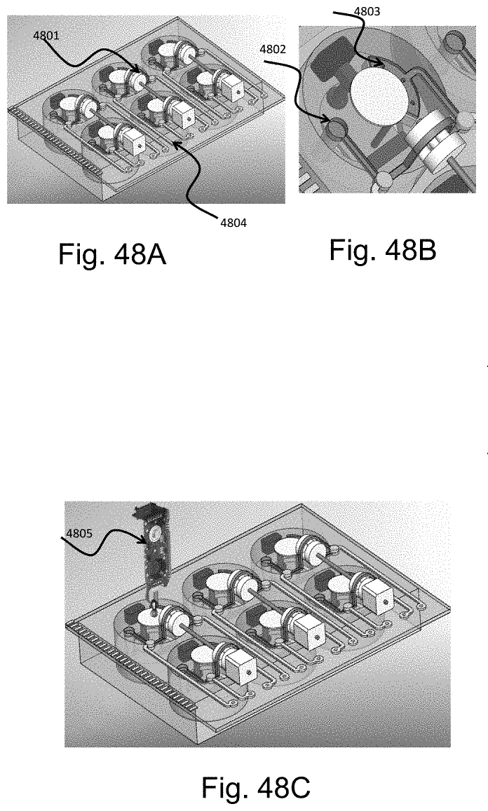

The system for electrical and mechanical stimulation of chip consists of a silicone based two layer 3-D inserts 4701 in 6-well plates for culturing muscle cells, a microfluidic chip for supplying media 4702 and drugs/reagents to the 6 wells 4703. The 3D inserts with top and bottom chambers capable of uniaxial mechanical stretching 4704 and electrical stimulation 4705 within 6-well plate as shown in FIG. 47A. Skeletal muscular cells embedded in fibrin gel are loaded on the top chamber and are cultured under perfused media with electrical stimulation and cyclic strain to replicate structure-functional relationships of native muscle tissue. The silicone chip is capable of mechanical stretching to a maximum strain of 20% at 1 Hz in uniaxial direction. Electrical stimulation is applied on two conducting rods 4705 4706 connected to the scaffold. Muscle fibroblast may be loaded on the bottom chamber to affect muscle cells phenotype and synthesis of extracellular matrix. The top and bottom chambers are separated using a polycarbonate 5 um filter scaffold so that fluid exchange will be accomplished while the cells will remain in the chamber. A set of electromagnetic linear actuators 4707 are capable of providing mechanical stimuli to the cells on the 3-D inserts as in FIG. 47B. A set of peristaltic pumps 4801 provide recirculation of fluidics (vertical fluidics) within top and bottom compartments as shown in FIG. 48A. One of the wells is magnified in FIG. 48B showing pipette 4802 to pull fluid from the bottom well and to dispense 4803 to the top well. Periodic recirculation of fluidics within top and bottom compartments are activated through a microcontroller. The fluidic chip with fluidic system 4804 (horizontal fluidics) is capable of microfluidic programming of drugs at different concentrations. The system is configured to perform stretching, perfusion, electrical stimulation and field potential signals acquisition and optical imaging. For electrical stimulation of the cell, we will connect 8 channel biphasic current stimulator developed using octal digital to analog converter and amplifier followed by voltage to current converter. The system will operate from a battery of derive power through gas sensor hole of the incubator. The actuators are selected to offer low power operation. Electrical connection to pumps, actuators and stimulus electrodes are accomplished using an edge connector on one another side. Programming of the control system and post-processing for statistical analysis will be performed using custom software. Further force measurements on the functional muscles are carried out using suspended force sensor 4805 and controlled to measure in Z axis and measured across entire 6-wells using XYZ stage. High precision Z stage is used. A program to control Z-stage using feedback from force sensor so that the force measurement tip will not puncture the muscle cellular system.

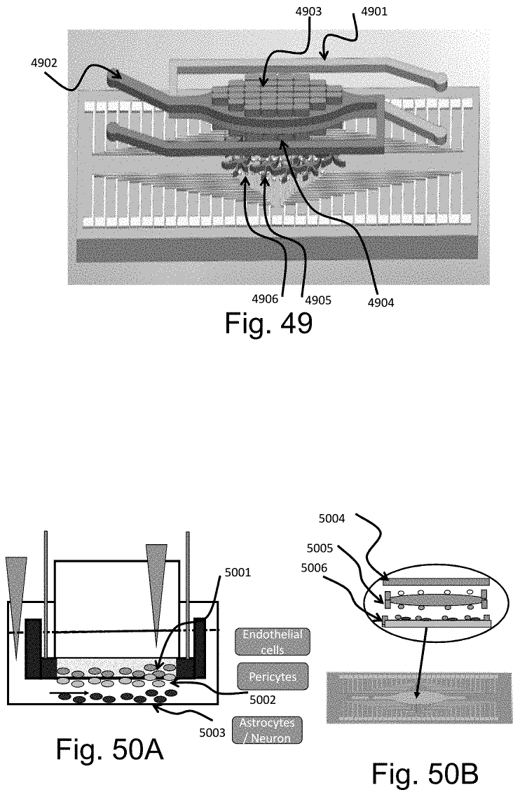

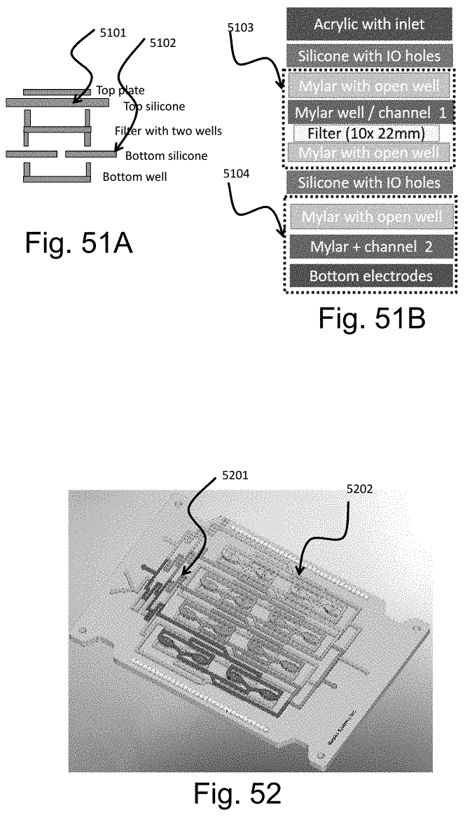

The system for blood brain barrier shown in FIG. 49 will perform neurological drug screening in microreactors using optical, TEER and microelectrode array field potential (FP) signatures. The chip (FIG. 49) consists of two sets of perfusion fluidics 4901, 4902 in two ellipsoidal reactor arrays separated by polycarbonate membranes of pore size 0.5 um-5 um. Human brain vascular endothelial cells 4903 are loaded in the top of the filter while Pericytes 4904 are loaded in the bottom of the filter and neuron and astrocytes 4905 are seeded on the electrodes 4906 in the bottom plate as in FIG. 50A. In FIG. 50B three pieces 5004, 5005, 5006 of the chip are shown. The chip consists of layers of fluidic channels and silicone sealing gaskets 5101, 5102 as in FIG. 51A. In order to fabricate the chip different layers of channel and wells are assembled in two blocks 5103, 5104 as in FIG. 51B. Further some of the parts can be fabricated at large scale using injection molding technique. The perfusion reagents such as drug and cell media are brought into cell reactors by in-situ pumps. In this chip, ˜1000 cells are loaded in each reactor and the top surface is sealed using a spring loaded manifold. Perfusion fluidic media with nutrients is carried out for several days to weeks. The system is configured to perform fluidics, cell stimulation and data acquisition from multiple microelectrodes. With our previous experience with gradient devices, perfusion fluidics and electrophysiological neuronal drug screening experiments, we will develop a high-throughput system. Initially, prototype chips will be fabricated using acrylic/glass substrate with ITO/platinum electrodes layers.

The chip consists of microfabricated electrodes on the top and bottom layers for TEER measurements. We have developed a custom circuit for multichannel impedance measurement and FP measurements from the bottom layer array of electrode sensors. After the fluidic experiments, the cells in the layered chip could be interrogated by other relevant assay modalities, such as to determine molecules that can potentially traverse via the transcytotic pathway, gene expression from the cells comprising BBB, immunohistochemistry after fixing cells. We will develop a high throughput system using a 24 well format for drug/combinatorial dose produced by a microfluidic gradient generator network 5201 and repeated reactors 5202 as in FIG. 52.

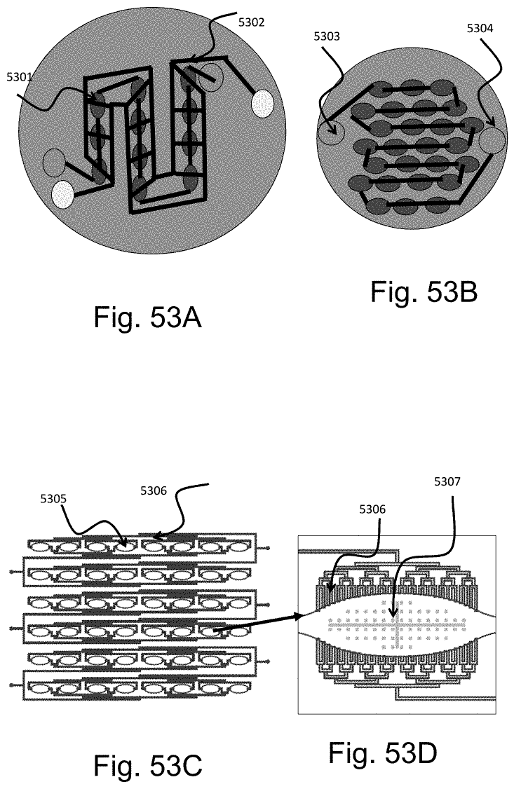

In order to develop cells and organs with vascular network cells in gel is seeded in a central channel 5301 while fluidic perfusion of media is performed in the outer channels 5302 as in FIG. 53A. The inlet 5303 and outlet 5304 of the chip (as in FIG. 53B) are connected to microplate tips so that pumping can be automatically performed using a perfusion control system in a 6-well format. These inserts are pluggable in the tips after loading the gel in the central channel. The gel loading channel input and output can be used for perfusion of the fluids in the automated perfusion system. In FIG. 53C, cells are loaded with gel in the wells, the top layer with perfusion fluidic channel fingers shown in FIG. 53D, will feed the cells with media. The bottom plate has multi electrode array for recording field potential signals.

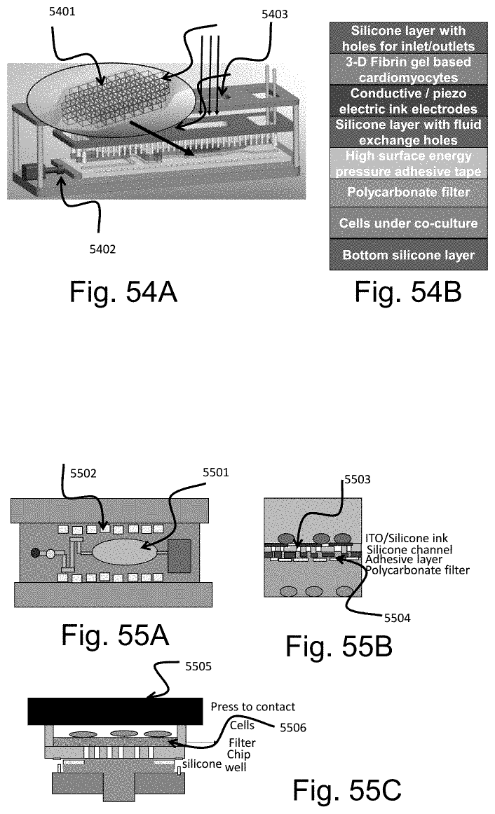

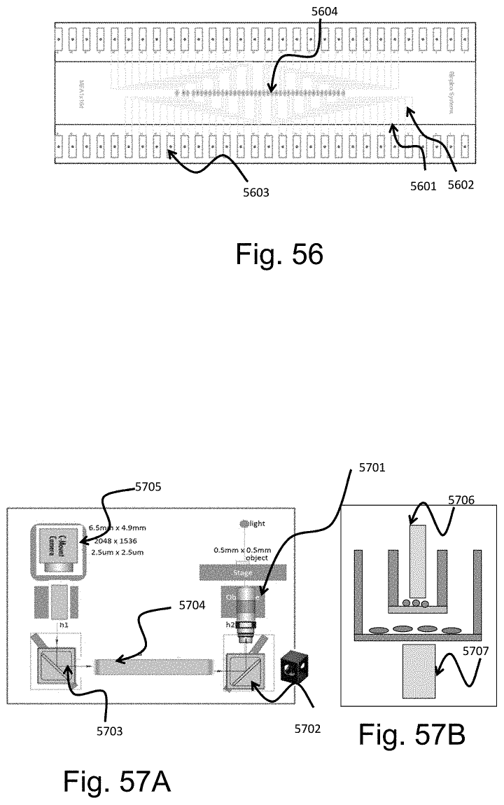

The chip for developing functional cardiomyocytes consists of a silicone based multilayer 3D microfluidic vascular chamber embedded with conductive ink electrodes and piezo-resistive electrodes capable of uniaxial stretching as in FIG. 54A. A four-head dispenser on XYZ liquid spotter for the tissue construction will fabricate the chip sensor. Electrical leads and contact pads are printed using a high-conductivity, silver particle-filled, polyamide (Ag:PA) ink (electrical resistivity of 6.6×10-5 Ohm cm), dilute thermoplastic polyurethane (TPU) inks, filled with 25 wt % carbon black nanoparticles (CB) form an elastic piezo-resistive material and viscous polydimethylsiloxane (PDMS) ink is used for vascular channel and insulator. The construction of the chip with different layers is shown in FIG. 54B. In FIG. 55A top view of the chip is shown. In FIG. 55B, side view of similar device is shown. In FIG. 55C the top layer is of the chip is connected to the chip by pressing against the chip so that fluidic leak proof operation can be performed. Cardiomyocytes (iPS derived) mixed with fibrin gel with extracellular matrix are loaded on chip and are cultured under perfused media with electrical stimulation and cyclic strain to replicate structure-functional relationships of native cardiac tissue. The chip has 16 recording electrodes for field potential recording and/or electrical stimulation. Field potential signals from the cardiac cells are amplified using a low noise amplifier array and data are acquired at 30 kSamples/sec/channel using our field potential measurement system. FIG. 56 shows micro array electrodes for one directional field potential measurements so that action potential conductivity measurements can be performed. The silicone chip is capable of mechanical stretching to a maximum strain of 20% at 1 Hz in uniaxial direction. The fluidic chamber is capable of microfluidic programming of media and nutrients through a fluidic manifold. An electrical fixture with spring loaded connector, driven by a linear motor, is used to contact electrical pads of the chip while not in mechanical stimulation. The cells seeded in fibrin gel are culture in the silicon vascular chip and perfusion of media/reagents will be carried out using the manifold. The mechanical stimulator is turned off for electrical stimulation or measurement by lowering the spring loaded connectors using another linear actuator from the top. The system is configured to perform stretching, perfusion, electrical stimulation and field potential signals acquisition and optical imaging. Programming of the control system and post-processing for statistical analysis will be performed for the quantification of performance metrics. Electrical and mechanical stimulation of the cells are carried out simultaneously and sequentially to study of the effect of the functional development of the cardiac tissue. The optical images and field potential measurements will be performed periodically in between stimulations. The simultaneous field potential recordings are analyzed for conduction and repolarization patterns of the cells. We will test the system for dose-dependent prolongation of the field potential duration using antiarrhythmic agents, and conduction slowing Na channel blockers. As many different ion channels contribute to the measured extracellular field potential, we will sort the recorded field potential spikes using wavelet transform and calculate field potential duration, conduction velocity and burst rate to provide effects on re-polarization of cardiomyocytes with stimulation parameters. We will measure piezo-resistive measurements to characterize mechanical stimulation parameters. The spike characteristics will be recorded for every dose and monitored every 3-24 hours. For the determination of statistical significance, 1-way ANOVA analysis followed by the Tukey's multiple comparisons test or Dunnett's post hoc test with appropriate control will be carried out. Evaluation of the functionality of the cardiomyocyes will be carried out using optical measurement as well as using field potential signals. With the functional cardiac cells, we will establish performance metrics using multiparameter statistical analysis. The chip with seeded cells is inserted in the system and pulse perfusion of media is carried out together with electrical and mechanical stimulation shown in

TABLE 1
Typical Stimulation Parameters from the literature

| | Type of | | | | Main result for functional |
|---|---|---|---|---|---|
| | Stimulus | Frequency | Amplitude | | muscle due to stimulation |
| Electrical | Bi-phasic | 1 Hz | 0.3 | V/mm | Tissue constructs generated |
| | pulses | | | | twitch force of 41.7 6 3.5 mN. |
| | Electrical | 1 Hz | 10 | V | Gene Expression fold |
| | pulses | | | | increased ~1-2 fold |

| | Type of | | Stretch/ | | Main result for functional |
|---|---|---|---|---|---|
| | Strain | % Strain | Time | | muscle due to stimulation |
| Mechanical | Static | 10% strain | 60 | mins | Lactate Concentration ~3 fold. |
| | and Ramp | | | | Ramp loading increased MMP-9. |
| | loading | | | | Stat ic IGFBP-5. Both static & |
| | | | | | ramp loading IGFBP-2 |
| | Uniaxial | 5-10% | 200 | um | Porosity of fibrin fibers ~1.82% |
| | | strain | | | and stiffness of fibers was ~3.30 |
| | | | | | kPa. Calcein intensity ~50% with |
| | | | | | 1.4 mN at 6% strain. |

### Cell Inserts for Well Plates.

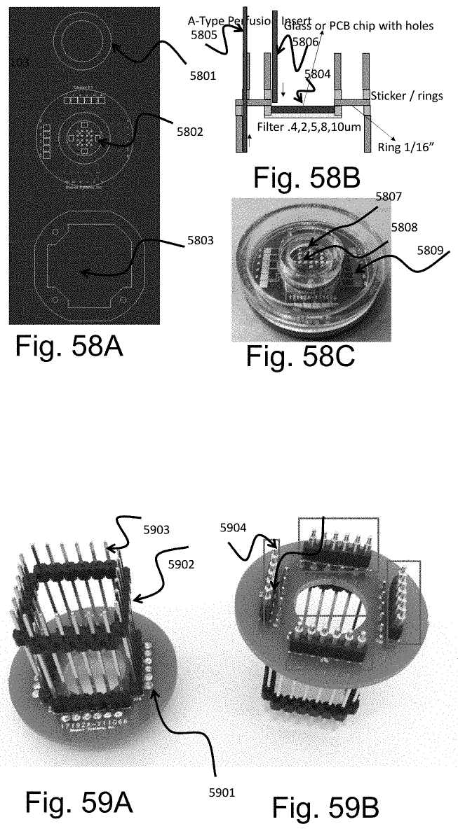

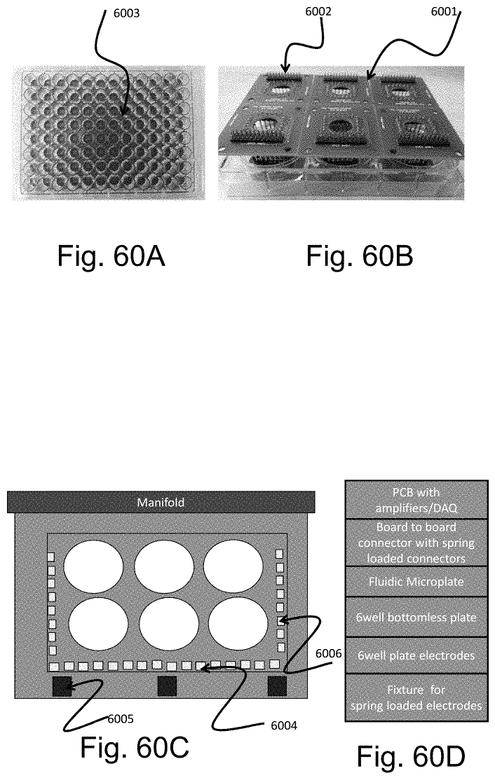

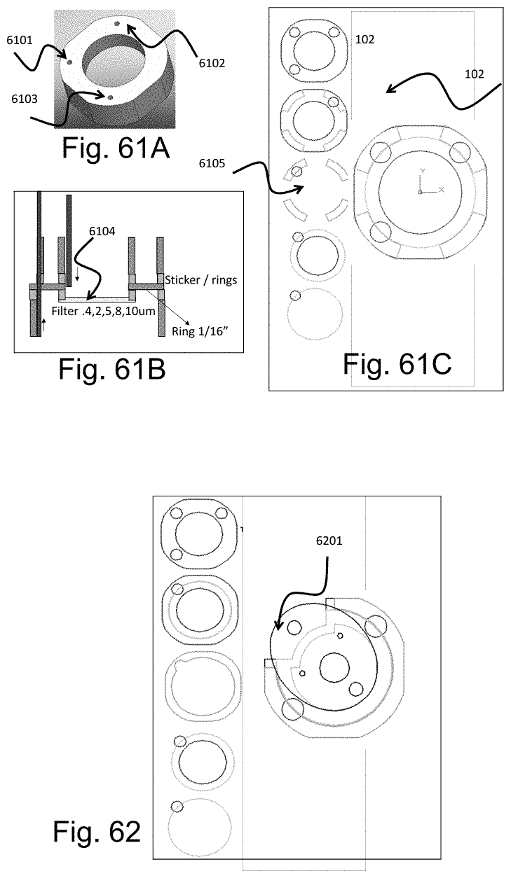

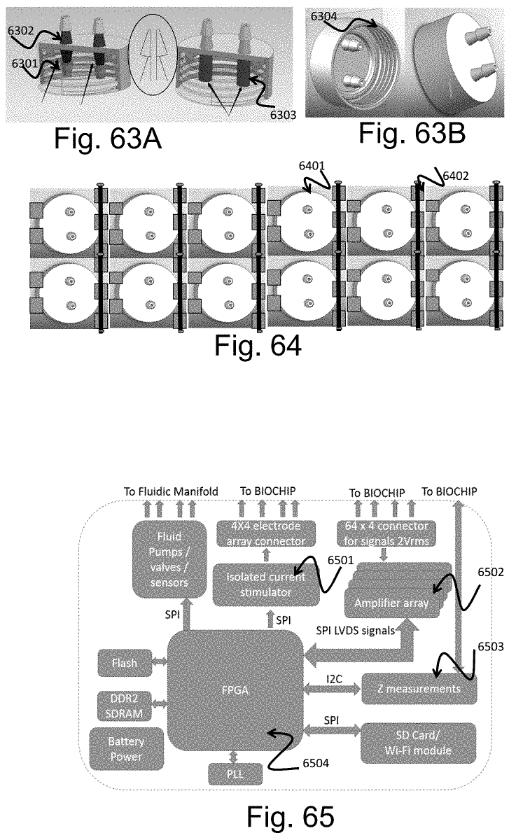

The well with insert is often used for 3-D cell culture. In these wells with insert, simultaneous imaging of the cells can be carried by a compact microscope as in FIG. 57A. It is important to image the cells on the top and bottom of a 3D insert and so a GRIN lens is used for the imaging on the narrow top well as in FIG. 57B. An insert for measuring field potential signals shown in FIG. 58A consists of top inner well, middle electrode layer with holes and bottom well that will go in a well plate. FIG. 58B shows the side view of the insert with holes or fluidic tips for pulling and pushing fluids. Alternatively fluid can also be pulled from inner well and dropped in to the outer well. FIG. 58C shows an insert with electrodes on a circuit board and holes for fluid exchange. There are electrode pads outside the inner well. This chip is fabricated in glass using ITO electrodes or modified with polyelectrodes or platinum, graphene or nanomaterials. The measurement system consists of spring loaded connectors (shown in FIG. 59B) on another intermediate PCB with extended connectors (FIG. 59A) to connect to a top PCB. The top PCB with all the electrode terminals (shown in FIG. 60B) is connected to amplifiers and field programmable gatted array for transmiting the signal to a central cloud server. We can also use similar approach to equip standard well plates with electrodes for field potential measurement as in FIG. 60A. In another design of perfusion system with field potential measurement, the electrodes pads are connected outside the wells as in FIG. 60C. A fixture is used to lock the sring loaded connectors from the measurement system to the electrodes pads as illustrated in FIG. 60D. Further the inserts with fluidic hole and alignment holes is fabricated using injection molding as in FIG. 61A. These inserts can be without any electrodes as shown in FIG. 61B to perform optical imaging based assays. Further the bottom skirt of the insert can be with discontinuous cyliners as shown in FIG. 61C so that it will provide channel for water to flow in to the bottom well. In the case of top pulling microplate, dispenser will drop fluid in to the bottom well while pulling fluid from the top well. the insert for this purpose is modified with provision of dispensing fluid in to the bottom well as shown in FIG. 62. Another aspect in the microfluidic perfusion system is the design of reservoirs with caps for fluid entry/exit ports. The ports in the cap requires locking tubes using barb connectors inside and outside the cap as shown in FIG. 63A. For easy fabrication straight connector is preferred. Such cap also need leak free closing when operation and so multiple screws are required to tighen up as shown in FIG. 63B. In order to cascade several reservoirs for delivering fluids and to receive used media, a method to lock the reservoirs together is shown in FIG. 64.

Electrical Instrumentation

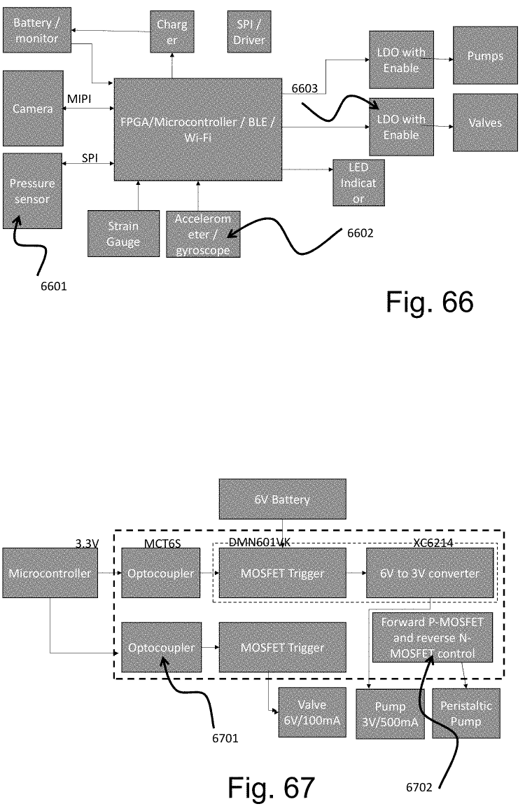

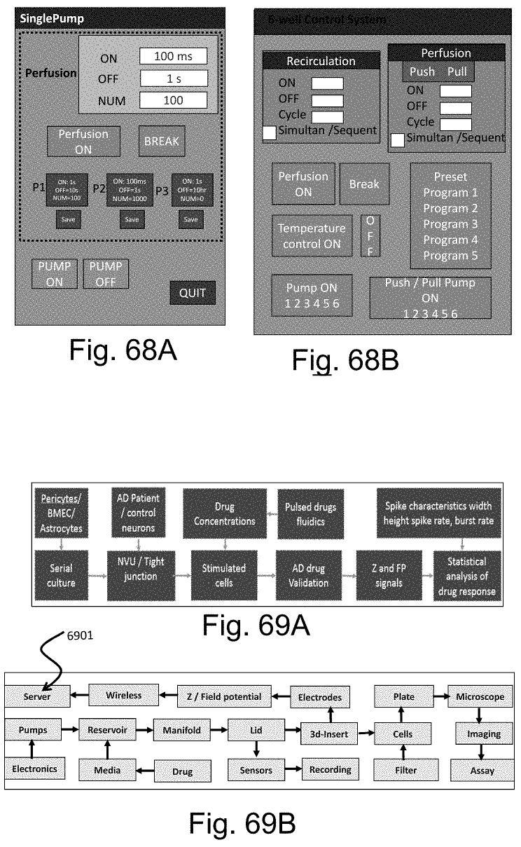

For electrical stimulation of the cells, we will use our 8 channel biphasic current stimulator developed using octal digital to analog converter (Maxim Integrated) and amplifier followed by voltage to current converter. Field potential signals from the cardiac cells are amplified using a low noise amplifier array and data are acquired at 30 kSamples/sec/channel using our field potential measurement system. Low voltage differential signals are handled through a converter for connecting to Field programmable gated array. In some cases impedance measurement for transepithelial electrical resistance (TEER) and label free cell proliferation measurements are measured. These signals are measured and transmited to the cloud as shown in FIG. 65. Further multiple signals such as pressure sensors for the fluidic circuit, accelerometer to sense any vibration of the system, gyrometer to measure angular velocities of roll, pitch, and yaw. Low drop out (LDO) linear regulators are used for controlling pumps as shown in FIG. 66. The communication to control devices are executed using BLE while high speed acquisition of data is carried out by WiFi. A MIPI interface is used for connected camera to Field programmable gated array. The system will acquired images from the cells and send to the cloud so that scientists can look at the images or data remotely. In order to interface microcontroller or Field programmable gated array with pumps or valves, a MOSFET trigger with an optocouple is utilized. In the case of flowing fluid using a peristaltic pump in the forward and reverse direction, both p-MOSFET and n-MOSFET is used as in FIG. 67. A control application software for single pump or multiple wells control system is developed as in FIG. 68A and FIG. 68B.

Protocols for Cellular or Organ Assay

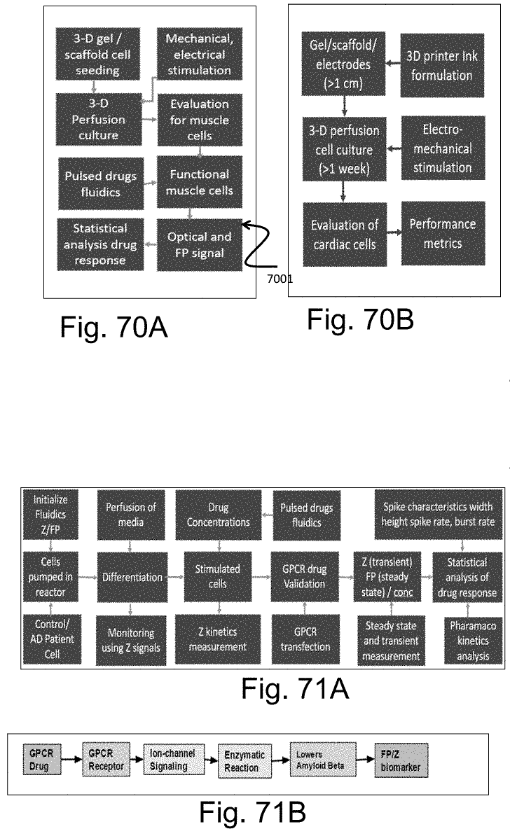

A general protocol for carrying out circulation, perfusion of media or drug or other reagents in to cell or organ is presented in FIG. 69B. Imaging, field potential measurements, impedance measurements and transmisson of data or images to a cloud server is performed. For a blood brain barrier assay, Human Brain Microvascular Endothelial Cells are used to form an artificial blood-brain-barrier using EBM-2 basal medium containing 5% Fetal Bovine Serum, 1% Penicillin-Streptomycin, 1.4 μM hydrocortisone, 5 μg/ml ascorbic acid, 1% CD Lipid Concentrate, 10 mM HEPES and 1 ng/ml basic fibroblast growth factor. Pericytes are loaded on the bottom side of the filter and cultured to adhere well. Later, the endothelial cells are loaded on the top reactor while astrocytes and neurons are cultured on the electrodes. We have developed a bioassay protocol, shown in FIG. 69A to record TEER and FP measurements. Study of functionalized muscle cells with electromechanical stimulation and optical imaging to account for force or stress on the cells due to elongation of sarcomers as shown in FIG. 70A. In FIG. 70B electromechanical stmulation of cardiomyocytes and measurement of optical imaging and field potential signals is presented.

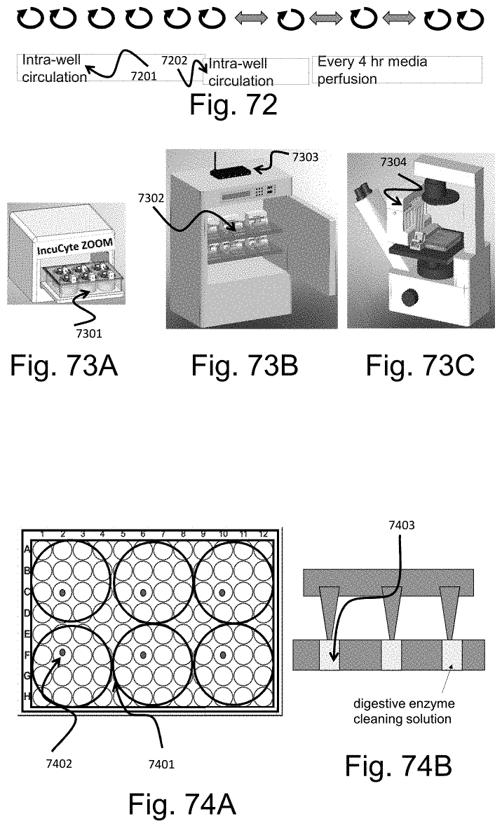

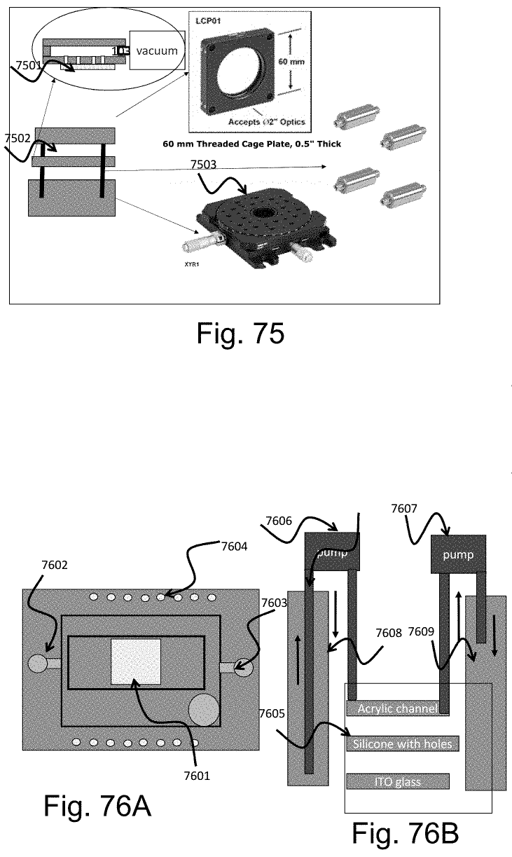

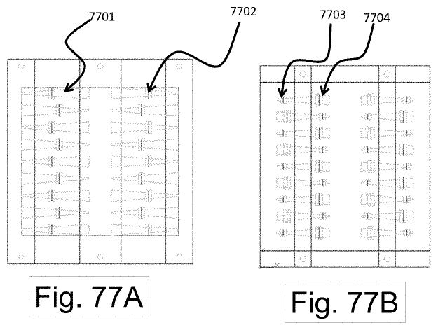

We have developed a protocol to study GPCR based drugs for Alzheimer's disease on neural cells for impedance differential measurement with dynamic flow conditions and field potential signals under steady state and transient flow conditions as shown in FIG. 71A. FIG. 71B shows the effect of GPCR drug on the ion channel signalixing that lowers amyloid beta which will be reflected in field potential or impedance signals. The circulation liquid pump can operate in forward and backward direction to provide intra-well and inter-well fluidic circulation. The perfusion air pump activates as pushing a measured volume of fluid as a pulse from the reservoir into the well and pulling the same volume from the well in to a waste reservoir. The lid can be custom made for several application or interacting-organs fluidics as shown in FIG. 7. In order to accomplish the fluidics of interacting-organs shown in FIG. 6a, the fluidic configuration shown in FIG. 7f will be developed where each organ is interacting to heart. In the case of organs system on well plate different organs can be connected through fluidic network through the microplate and fluidic pumps with reagents. The fluidic system provides fluidic programmable pumping in to the 6-well based organ system. In the case of the interaction between heart and liver in closed-loop circulation that mimic physiological phenomena, the protocol to circulate media and their metabolites within well and inter-wells is presented in FIG. 72. Both liver and heart play a major role in metabolic activity and blood circulation in body homeostasis, and so we have selected this system for our study. The drug effects on this system will serve as a killer experiment for clinical trials. The system will be modified to fit in commercial incubator and imaging system such as Incucyte Zoom as in FIG. 73A. Several of the systems can be accommodated in an incubator. Monitoring of the cells and organs for impedance, field potential signals are carried out remotely through WiFi interface as in FIG. 73B. The system can be held in a microscope for imaging the cells or organs with temperature and hypoxia control as in FIG. 73C. In order to clean the fluidic system for culture, a cleaning plate as in FIG. 74A with 6 holes fitted with 6 vials of 2 ml volume is used. The vials are filled with digestive enzyme cleaning solution or 70% ethanol and aligned to the 6 tips of the microfluidic plate as in FIG. 74B. A cleaning program is selected in the smart device control for cleaning the fluidic system. In order to assemble the inserts or microplates a vacuum sucker and linear aligner and rotation al aligner will be used as in FIG. 75. For electroplating the electrodes for field potential measurement or impedance measurement, a flow based system shown in FIG. 76A is used. The system will have inlet and out and hold the electrode chip using spring loaded connector. A detachable fluidic well or channel is pressed under silicon layer on the electrode substrate as in FIG. 76B. Two set of pumps were used to flow electroplating fluid from fresh reservoir through the chip to a waste reservoir. Further a protocol for fabricating tips for microplate is developed using laser or mechanical cutting from an array of tips on a template holder as in FIG. 77 A and FIG. 77B. The tips are cut at one place or places in order to form a repeatable tip height and diameter.

Validation Using Cells and Drugs

In order to ensure that the perfusion system is adapted for clinical studies, we will design experiments to perform under GLP. Assessment of various cardiac drugs and combinations including excitatory and inhibitory drugs will be tested. Once assay parameters and range are set during the assay development, we will design limited experiments to show linearity, accuracy, precision, specificity, robustness, ruggedness and system suitability for assay validation. Evaluation of the functionality of the cardiomyocyes or skeletal muscles will be carried out using optical measurement from the Incucytes. After the cells will be attached to the chip, may take 48 - 72 hours with media perfused for every 3 - 12 hours. The cells will be maintained with a constant cyclic strain (20%, 1 Hz) and electrical stimulation (0.2-0.5 mA, 2-5 Hz) before or after the measurement periods. The imaging of the cells will be performed periodically while turning off stimulations. The AD hIPS derived NSC, control hIPS derived NSC and AD hIPSC derived NSC that will be procured for the validation study. Electrophysiological and genomic characterization of these cells are compared with perfusion and without perfusion. We will explore several drugs such as donepezil, galantamine, memantine and rivastigmine for AD. We will study the effect of the drug dosage on the cells using Doxorubicin and Valproic Acid. The effect of drug toxicity on the liver cells are measured using an immunoassay from sampled media from the well over a period of 14 days. In order to perform the feasibility study, human immortalized skeletal muscle myoblasts (ABM Cat.No.:T0033) will be seeded in Fibrinogen and Matrigel mixture for 3D culture. 3T3 fibroblasts from Lonza will be culture at the bottom chamber. The cells under cyclic strain and electrical stimulation will be characterized using imaging for live cell morphological analysis. The drug study will be carried out for sarcopenia using anamorelin drug for ghrelin-receptor agonist and will be validated for a EC 50 of 15 nM (IC50=0.21 uM). In order to perform the feasibility study, iPS derived Cardiomyocytes will be seeded in Fibrinogen and Matrigel mixture for 3D culture. The cells under cyclic strain and electrical stimulation will be characterized for live cell morphological analysis through microscopic imaging. We will test our system for dose-dependent prolongation of the field potential duration (FPD) using class I (Quinidine, Procaineamide) and class III (Sotalol) antiarrhythmic agents, and conduction slowing Na channel blockers (Quinidine and Propafenone). The effects of increasing concentrations will be studied using Sotalol (10-400 μM), Quinidine (0.2-8 μM) for FPD and Quinidine (10- 200 μM) and Procainamide (3-120 μM) for conduction. To evaluate the effects of interaction between liver and heart through their metabolites, anti-cancer drug DOX was used as a model drug. Seven or fourteen days after the co-culture, cardiac beating frequency was quantified from video recordings of the cardiomyocytes culture. The inserts are coated with matrigel and cardiac and liver cells are seeded to culture at 37° C. incubator for organ interactions study. In order to perform the feasibility study, human hepatocytes (HepG2) and primary human cardiomyocytes (hCM) are chosen as model cells. The system for electrical and mechanical stimulation of chip consists of 6 well plates with 3-D inserts for culturing organs, a set of reservoirs to draw fresh media and drugs and to collect waste, a microfluidic lid to divert fluids from reservoirs to 6-well plates, a manifold to provide fast replacement of lids with a pumping system.

### EXAMPLES

### Example 1

### Electro-Mechanical Bio-Engineered Drug Screening (EMBEDS) System for Musculoskeletal Tissue Models

Several models to engineering of skeletal muscle constructs embedded in a fibrin scaffold under 3D cell culture with different strain regimes like static, cyclic or ramp strain have been developed to achieve muscle functions. However, biomimetic functional muscle in terms of organized muscle bundles structure, gene expression profile and maturity is still one of the fundamental challenges in skeletal muscle tissue engineering. Limitations such as high cost, extensive culture time and lack of functional skeletal muscle tissue, forbid the development for next generation therapeutic treatments. Therefore development of a simple, cost effective automated 3D culture system with electrical and mechanical stimuli capabilities to achieve functional skeletal muscle that can be screened with multiple concentrations of drugs is an unmet need for the clinical and research communities. In this regard, Biopico Systems develops an “Electro-Mechanical Bio-Engineered Drug Screening (EMBEDS) System for Musculoskeletal Tissue Models”. This automated fluidics and integrated stimuli drug screening system embedding skeletal muscles in fibrin gel for 3-D cell culture will be adapted to 6-well plate for routine drug screening applications. This in-vitro system aids in the testing of novel drugs and therapeutics to combat different treatments for genetic diseases such as muscular dystrophy, skeletal muscle injuries to replace and/or restore the damaged tissue and other anomalies that prevent skeletal muscle repair. We develop a prototype EMBEDS system adaptable to a commercial optical imaging system with established software for drug screening applications. The integration of our early stage device with a commercial system will allow to introduce the system to the scientific community much earlier, and the feedback can be incorporated into the final stand-alone system. Skeletal muscles, comprising ˜40% of a human body mass, are responsible for generating forces of voluntary movement and locomotion. Maturation of these muscle cells in 3-D culture is accompanied by an increase in contractile force of the myofibril, which is actuated through relative movement of thin actin and thick myosin filaments. The EMBEDS system enables automated and longer cultivation periods of muscle tissue with different stimuli applications and yield 3-D tissue engineered muscle with improved characteristics in regard to functionality and biomimicry. Further, the system is envisioned to provide understanding of endogenous healing cascades in clinically demanding situations such as treatment of skeletal muscle trauma and to stimulate vascularization and neurogenesis in regenerating muscles. Moving from the inside out, skeletal muscle is composed by myofilaments, sarcomeres, myofibrils, muscle fibers, and fascicles. Mechanical stimulation facilitates myoblast differentiation into a highly organized array of myotubes with widespread sarcomeric patterning and increased diameter compared to non-stimulated constructs. The alignment of cytoskeletal proteins and ECM components parallel to the axis of applied strain helps the cells adhering to a matrix of extracellular proteins to transmit the force to the cytoskeleton. Further to note that without proper electrical stimulation, muscle will atrophy and die and the contraction of a muscle tissue in 3D cell culture due to neuronal activity can be mimicked by applying an electrical stimulus. For example, early electrical stimulation accelerates the maturation of the tissue causing cross striations whereas cultures without electrical stimulation are slower. The regime of electrical stimulation such as duration, voltage, amperage, and timing plays an important, role in muscle differentiation. EMBEDS system integrate stimulation with fluidic perfusion in a portable format so as to reside in an incubator to provide continuous live-cell monitoring and analysis. In such environment, the cells are not disturbed and so repeated measures over time provide powerful insight into the time course of biology and provides greater control over critical assay conditions. The integration of the early stage EMBEDS system with a commercial imaging system will allow to introduce the system to the scientific community much earlier, and the feedback can be incorporated into the final stand-alone system. Further, using state-of-the-art kinetic analysis software built within the system, morphology of the cells, contraction ability, proliferation rate, presence of intercellular adhesion structures, organization of myofibrils, mitochondria morphology, endoplasmic reticulum contents, cytoskeletal filaments and extracellular rnatrix distribution, and expression of markers of muscle cells differentiation under co-culture of cells can be studied in order to characterize the EMBEDS system. Table 2 shows the rational for the key biological variable for the electrical and mechanical stimulation of the cells under culture.

TABLE 2
Key biological variables and biological significant/rationale for

| | Biological | | Measurement/ |
|---|---|---|---|
| Stimulation | Outputs | Biological Significant | Quantification |
| Electrical | Production of | Coordinated Contraction | Western Blot and |
| | Sarcomere | and Elongation for | Histology Staining |
| | Proteins | Functionality | |
| | Alignment of | Improved Contractibility | Calcium Imaging and |
| | muscle filament | and Differentiation | field potential |
| | (length/angle) | | measurements |
| | Myogenic Gene | Multiple Functions such as | Western Blot and RT- |
| | Expression | Survival, Proliferation rate | PCR and Immunoassay |
| | | and Adhesion | |
| | Nox, Ca2+ Release | Molecular Activator of | Electrochemical, |
| | in Culture | Satellite cells, membrane | fluorescence imaging, |
| | | potential | field potential signals |
| Mechanical | Organization of | Increased Contractibility | Western Blot and |
| | Myofibrils | Function | Immunoassay |
| | Sarcomere | Muscle Functionality | Immunoassay |
| | Proteins | | |
| | Alignment of | Muscle Hypertrophy | Western Blot Protein |
| | Muscle Filaments | increases cell tissue size | Isolation Assay, action |
| | | | potential |

### Example 2

### Parallel Neurovascular Electrophysiological Assay for Alzheimer's Disease Research

Alzheimer's disease (AD), a progressive degenerative disorder of the brain, affecting 40 million individuals worldwide burdens tremendous socioeconomic cost. This necessitates a global effort to better understand several processes in the neurovascular unit (NVU) against disruption, transporter dysfunction and altered protein expression and secretions. Because of the growing aged population an early treatment to prevent pathogenesis of AD is an urgent requirement. With the advent of patient-derived induced pluripotent stem cells for AD, there is a huge opportunity for not only studying disease pathogenic cascades but also for drug discovery. However, it has been challenging for commercializing the AD brain in-vitro models for clinical applications. Therefore, Biopico Systems Inc proposes to develop a Parallel Neurovascular Electrophysiological Assay

(PSEA) suitable for predicting therapeutically useful drug passage across the NVU relevant for the drug screening of AD in 3D culture. This proposed microfluidic AD pathogenesis on a dish with electrophysiological functional assay, has a great potential to be commercialized for clinical and pharmacological applications. We will validate the system using stem cells derived AD patients cell lines with excitatory or inhibitory drugs that will form the basis of establishing a clinical screening platform. This PSEA system has the potential to accurately and systematically evaluate the cellular mechanisms that disrupt the functioning of NVU in AD and to accelerate discovery of new AD drugs. The AD market is expected to rise to $5 billion in 2021, at a global CAGR of 7.9%. US pharmaceutical research companies are investigating around 100 medicines to help 5 million patients living with AD. Therefore the PSEA system has tremendous market allowing the evaluation of different pharmacological pathways and dosages in the development of anti-AD drug candidates. Further the system can easily be adapted to analyze other CNS disease-relevant targets to provide high throughput and reliable screening of drugs using neural stem cells.

Many cell types in addition to brain endothelial cells contribute to the essential function of NVU, including pericytes, microglia, astrocytes, neurons and the extracellular matrix proteins. Alzheimer's disease is caused by several dysfunctions of this NVU such as leakage of circulating neurotoxic substances into the CNS, inadequate nutrient supply, buildup of toxic substances, and increased entry of compounds that are normally extruded; and inflammatory activation, oxidative stress, and neuronal damage. Looking at specific genetic targets, amyloid precursor protein (APP) and the presenilin 1 or presenilin 2 mutation are associated with the downstream hypothesis effects of amyloid beta and tau accumulation. By using these genetic mutations to create a model cell line of the disease along with specific targeting of receptors that affect the downstream pathology of the disease, efficient and effective drugs can be researched. Thanks to recently advances in iPS cells an in vitro representation of their neural cells can be made and tested for responses to particular drugs, from any given patient by comparing diseased cells to normal ones pharmacologically. Our NVU drug screening system will improve the approval rate of AD drugs that will help us to commercialize for several clinical applications as in Table 3.

TABLE 3
Clinical Applications of BBB

| Clinical | | | |
|---|---|---|---|
| Applications | Drug | Examples | Measurement |
| BBB Diseases | Cilostazol | Intracranial Hemorrhage | Collagenase |
| | | Drug analysis | damage |
| Repeated drug | Colchicine | Safety evaluation of | P-gp |
| doses | | toxicity over time | functionality |
| Drug delivery | Cediranib | Delivery of anticancer | Permeability |
| | | drugs to treat | |
| | | glioblastoma | |
| Disease | Curcumin | Amyloid Beta | Levels of Aβ |
| modeling | | Degradation | |
| Drug design | Taxol | brain homeostasis | Transport ratio |
| Infectious | Chloroquine | Inhibition of Zika | ZIKV-induced |
| diseases | | Virus infection | cell death |

Integrated fluidic programming, electrophysiological monitoring and wireless data transmission system for drug screening in disease model will lead to establishing Good Laboratory Practice protocol reducing any sample movement out of the incubator, human error or any contamination in the assay protocol. Further, functional assay for AD is developed using integrated multi-electrode array based assay to monitor the electrophysiological properties of diseased and healthy neurons and their responses to potential therapeutic agents. Thirdly, dose or combinatorial drug dependent efficacy of therapeutic drugs, is addressed by establishing a fluidic scheme for serial drug concentration profiling by pulsatile homogeneous fluidic mixing. In this proposal we will apply this screening technique to iPSC derived AD cell model as a module to establish a protocol for clinical testing. Several past static models of the NVU did not mimic accurately due to lack of flow and shear stress needed to accurately represent 3D culture. In order to perform 3D cell culture and electrophysiological analysis of high-throughput samples, the PSEA technology involves integrating various engineering techniques. Using this PSEA system, complex assays can be performed with lower reagent consumption, in an automated, integrated and user-friendly system. This revolutionary system as compared in Table 2 will change our current paradigm of 3D cell culture, and evaluation by automatically conducting the sequential processes through custom-made instrumentation and software as a portable instrument.

### Example 3

### Fluidic Programmable GPCR Assay (FPGA) for Mental Health Disorders

Integrated and automated microdevices to elucidate the function of GPCRs and to identify selective agonists/antagonists have the potential to impact the future of GPCR-based drug screening. In this regard, programmability to precisely control fluid transport for rapid and homogeneous drug distribution and the ability to exchange buffers for agonist exposure control and receptor functional recovery in cell based assays will provide huge benefits in the advance of GPCR based drugs. Such drugs have great significance in healthy mental function and in mental disorders and therefore additional electrophysiological measurement in the screening of such drug interaction with neuronal cells will bring a paradigm shift in pharmacological validation. With the advent of patient-derived induced pluripotent stem cells, a unique opportunity to explore such assessment of the effects of these drugs in personal medicine for neurological diseases or disorders, is practical. However, presently, these static tests are slow, costly and wasteful and provide only a limited estimation of human response to chemicals for such in vitro “disease in a dish” models. We develop “Fluidic Programmable GPCR Assay (FPGA) for Mental Health Disorders” to provide programmable and reliable screening of GPCR drugs using diseased neural stem cells. In this proposal, the development of the FPGA system will provide smaller low reagent multiple step dynamic assay to perform different doses drug stimuli and to monitor in transient and endpoint electrophysiological assays. In this device the processes of liquid dilution, micro-scale cell culture, electrophysiological monitoring are integrated into a single device to automate entire drug screening protocol for the clinic. This FPGA system has the potential to provide patient-specific pharmacology information for diverse cellular responses of drug cocktails and to promote the understanding of disease pathology that disrupt the functioning of nerve cells. As a case study, in this proposal, we will validate the system using GPCR receptors transfected iPS derived cell lines AD patients and isogenic AD cell model from commercial sources with excitatory or inhibitory drugs that will form the basis of establishing a clinical screening platform for clinical pharmacology.

More than 50% of all current drugs and nearly 25% of the top 200 best-selling drugs target G-protein-coupled receptors (GPCRs). The FPGA functional assay system could be used as a routine tool for drug discovery for GPCR based drugs for neurological diseases. This sensitive measure for detecting GPCR response provides pharmaceutical information for high throughput and reliable screening of drugs using neural stem cells. GPCRs represent the largest therapeutic target in the pharmaceutical industry GPCRs are found to be approximately 90% expressed in the brain and involved in many processes such as cognition and synaptic transmission and several GPCRs are involved at many stages of neurological disease progression. Drugs that target GPCRs could diversify the symptomatic therapeutic portfolio and potentially provide disease modifying treatments¹²⁻²⁷. For example, numerous drug discovery efforts target the inhibition of amyloidβ production, the prevention of amyloidβ aggregation and the enhancement of amyloidβ clearance in Alzheimer's disease. GPCRs can modulate ion channel activity through an indirect pathway that involves a common second messenger leading to the phosphorylation of the channel or through a direct pathway, involving binding of Gβγ directly as membrane delimited modulation. Therefore establishing electrophysiological based biomarker is a significant step in the drug screening using GPCR. Progress in the GPCR drug discovery is hampered by the difficulty in developing highly receptor specific ligands and the adverse side effects of currently available drugs. Microfluidic dynamic invitro assays²⁸⁻³⁰ for thousands of GPCR drugs with electrophysiological screening of cells provides a paradigm shift in predicting pharmacological response in neurological diseases or disorders. The efficacy of therapeutic drugs, as well as interaction between different drugs, is dose-dependent and so integrating processes of liquid dilution, micro-scale cell culture, electrical impedance (Z) and field potential (FP) measurements into a single device to automate entire drug screening protocol can accelerate clinical applications. Functional approach towards the structural classification of GPCRs, would enhance the therapeutic potential of GPCRs. Therefore, the FPGA system (as in FIG. 1) will perform drug screening using GPCR transfected iPSC derived AD model cells³¹⁻³⁴ and monitor using multiple modalities (Z, FP, optical) to establish a protocol for pharmacological/toxicological testing. An integrated multi-electrode array-based assay to monitor the electrophysiological properties of diseased and healthy neurons and their responses to potential therapeutic agents is highly significant as a functional assay for GPCR drugs. In order to perform cell culture, cell differentiation and electrophysiological analysis of high-throughput samples, the FPGA technology involves integrating various engineering techniques such as microliter scale iPSC differentiation protocols, wireless data transmission and electrical signal conditioning and analysis. Using this FPGA system, complex assays can be performed with lower reagent consumption, in an automated, integrated and user-friendly system. This revolutionary system, as compared in Table 1, will change our current paradigm of cell culture, NSC differentiation, and evaluation by automatically conducting the sequential processes through custom-made instrumentation and software as a portable instrument.

### Example 4

### Regenerative Electromechanical Aided Chemical Stimulation with Transducers for Opto-Electrophysiological Recordings for Cardiac Pharmacology

Biomechanical, electrical and chemical stimuli play a vital role for normal cardiac development and are shown to activate signal transduction pathways and subsequently regulate cardiac functions. Such stimuli in 3-D cellular culture influences morphology, contractibility, proliferation, adhesion, organization and gene expression and exhibits in vivo hierarchical structure, cellular interaction, diffusion barriers and cellular heterogeneity. In this regard, our ability to modulate cellular biochemical reactions would help in the development of functional drug screening applications. In order to assess the potential efficacy of a new compound in drug discovery, using induced pluripotent stem cells, the differentiated myocardium should display highly organized sarcorneres, cellular junctions, and an extracellular matrix surrounding the cardiac cells in 3-D cell culture. Therefore, there is an urgent clinical need to engineer functionally viable regenerative tissues using stress parameters that mimic the native environment. Such model systems with externally applied forces will not only further our understanding of therapeutic approaches to cardiac regeneration but also would enable to develop a drug screening function assay for cardiac diseases. Therefore Biopico Systems Inc develops “Regenerative Electromechanical Aided Chemical stimulation with Transducers for Opto-electrophysiological Recordings (REACTOR) to support cardiac pharmacology”. This REACTOR system will be developed at Biopico Systems Inc and validated in a GLP regulated environment for pre-clinical and subsequent clinical adaptation. The REACTOR system will be established as inexpensive, easily manipulated, easily reproducible, physiologically representative of human disease, and ethically sound system. In Phase we will develop a prototype REACTOR system to be adaptable to a commercial optical imaging system for drug screening applications. Such system will provide complementary features such as electro mechanico chemical stimulations capabilities and electrophysiological monitoring in a fully automated fashion. With revenues and experiences gained from the add-on device, we will further our development to high throughput independent system for drug screening.

Cardiac cells can be mechanically and electrical stimulated by tensile, compressive, or cyclic strain which influences a number of cellular phenomena. Such understanding of how cells respond to stimuli is a critical step in learning how to direct cells in vitro to develop drugs or cells or regenerative tissues for cardiac applications. The global drug screening market is expected to see total sales of US$6.3 Billion by 2019. The REACTOR system can contribute to this market by establishing an innovative drug screening platform that will stimulate and monitor cells in functional assay for long time. For example, the system can access the potential efficacy of different antiarrhythmic compounds as well as determine the potential pro-arrhythmic risk of other pharmacological agents. The platform will help to identify any potential drug failure as early as possible and to avoid higher costs and efforts. A cell on bioreactors is an adaptive mechanical structure that both receives and responds to biochemical, biomechanical, and bioelectrical signals. Further mechanical stimulation of cells results in cell-generated responses for a variety of cell processes including differentiation, proliferation, extracellular matrix production, alignment, migration, adhesion, signaling, and morphology. During cardiomyopathy, Tgf-β signaling is thought to activate resident cardiac fibroblasts, leading to excessive fibroblast proliferation, cardiac fibrosis, and stiffening of the heart through excessive deposition of extracellular matrix. The high-throughput multi-electrode array-based assay to monitor electrophysiological properties cardiac cells and their responses to potential therapeutic agents is highly significant in that it allows the establishment of an assay for personalized drug selection. The field potential spikes, firing rate measurements can predict the effect of drugs on both repolarization (QT screening) and conduction properties of cardiomyoctytes. For example, ionic currents governing cardiac repolarization characterize drug-induced prolongation of the QT interval associated with arrhythmogenesis and slowing of conduction, caused due to reduction in excitability and decrement in cell-to-cell coupling, is an indication of reentrant arrhythmias. During continuous live-cell monitoring and analysis, cells are not disturbed by the observation and analysis and so repeated measures over time provide powerful insight into the time course of biology and provides greater control over critical assay conditions. The integration of the early stage device with the Incucyte ZOOM system will allow to introduce the system to the scientific community much earlier, and the feedback can be incorporated into the final stand-alone system. Further, using state-of-the-art kinetic analysis software built within the system, morphology of the cells, contraction ability, proliferation rate, presence of intercellular adhesion structures, organization of myofibrils, mitochondria morphology, endoplasmic reticulum contents, cytoskeletal filaments and extracellular matrix distribution, and expression of markers of cardiac differentiation can be studied in order to characterize the REACTOR system. Table 1 shows the rational for the key biological variable for the electrical and mechanical stimulation of the cells under culture.

Our overall goal involves integrating various engineering techniques such as concentration gradient fluidics, fluidic perfusion and nanoliter scale iPS cell differentiation protocols, electromechano stimulation and electrical signal conditioning and analysis. Such assay in a perfusion format fitted with microfluidic channels will consume only microliter to nanoliters of reagents, Using this REACTOR system, complex assays can be performed with lower reagent consumption avoiding cell contamination and adaptable to GMP/GLP and providing highly parallel operation in an automated manner. This revolutionary system will change our current paradigm of 3D cell culture, stimulation, and evaluation by automatically conducting the sequential processes through custom-made instrumentation and software as a portable instrument. Table 2 compares the REACTOR technology with existing competitive methods to bring the advantages and features of REACTOR system.

### Example 5

### Micro-Physiological Interacting-Organs Preclinical In-Vitro (MIPI) System for Drug Development

Human on a chip systems with interaction of multiple organs provide in-vivo tissue-like realistic cellular behavior environments and provide information on quantitative, time-dependent phenomena when combined with pharmacokinetic modeling approach. These improved interacting-organs assay with human cells is viewed as a next generation in-vitro platform alternate to conventional animal tests and preclinical drug development. However, the current in-vitro organs technology is still insufficient to match the complexity of the human body and development of multiple tissues, each of them having multiple cell types typically in a complex architecture is still in its infancy. This under development is largely due to the lack of suitable sterile instrumentations to provide interaction among different organs via a circulation system similar to human body. Although several microfluidic systems have been attempted to develop such multi-organs systems, they are too complicated for both researchers and pharmaceutical industries to handle their organ model. While large-volume circulation system does not take advantage of miniatured microfluidic device, present microfluidic chips are inconvenient for researchers currently working with standard well formats. Therefore, Biopico Systems Inc, develops a Micro-physiological Interacting-organs Preclinical In-vitro (MIPI) system to take advantage of microfluidic fluidic circuits and cell culture in standard well format. This enables recreating organs interactions by medium perfusion, inter-well and intra-well recirculation and evaluating drugs by monitoring multiple organs simultaneously. We develop our platform for 6-organs culture and demonstrate the feasibility for the interaction between liver and heart that mimic physiological phenomena for more accurate drug screening and safety testing. MIPI will be adapted by the pharmacological industries and researchers for testing drugs with unknown metabolic property and gain broader use for pre-clinical drug safety tests. There were 2.3 million reports of adverse drug effects submitted to FDA across 6000 registered compounds between 1969 and 2002. Consequently, 75 drugs or drug products were removed from the market due to these unpredicted effects. A significant proportion of these compounds validated during preclinical trials have unpredicted problems during human clinical trials. The MIPI system enables automated and longer cultivation periods for testing these compounds in interacting-organs for more accurate drug screening. This MIPI system together with refined models of interacting-organs system will improve the predictive power of preclinical safety testing and provide significant benefit to pharmaceutical industry to generate safer human-specific compounds.

It is estimated that only one in nine drug candidates that enter clinical testing reach the market, indicating therapeutic drug development needs more versatile, informative, and rapid pre-clinical models and accurate prediction of human safety and efficacy. In this regard, interaction among different organs under culture should be simulated like circulation system in a body enabling organ functions as coupled system, e.g., heart: volume pumped; lung: gas exchanged; liver: metabolism; kidney: molecular filtering and transport; brain: blood-brain barrier function. This development of interacting-organ systems capable of reproducing the functionality in a quantifiable manner for prediction of human tissue behavior is an unmet need. However, current efforts lack the dynamic flow of nutrients and toxins generated in living systems for extended time periods (>7 days) and system capable of providing interacting-organ environment in traditional well formats. This provides an immense opportunity for Biopico Systems to develop a Micro-physiological Interacting-organs Preclinical In-vitro (MIPI) system. MIPI system integrate fluidic perfusion in a portable format so as to reside in an incubator to provide continuous organ interaction and capable of adapt to a microscope environment for valuable optical imaging. MIPI system will validate body-on-a-chip systems as models for repeated dose or chronic exposure of compounds for efficacy, toxicity and pharmacokinetic studies. In this system, viable and functional human cardiac, liver, and other cultures within a common defined medium can be cultured for more than two weeks to provide insight into important metabolic and functional changes in human tissues in response to challenge with compounds with well-defined toxicological properties. Conditioned media sampled from specific tissue types of interest in compartmentalized organs culture in order to analyze their metabolites and other secretory products may aid in the identification and development of novel biomarkers for efficacy, toxicity or disease processes. MIPI system can appropriately provide flow rate requirements to both central compartment viewed as a lumped sum of rapidly-perfused tissues (liver, kidney, heart, and lung) and peripheral compartment viewed as a lumped sum of slowly-perfused tissues (muscle, fat, and skin). Therefore, MIPI system enables the reconstitution and visualization of complex, integrated, organ-level responses not normally observed in conventional cell culture models or animal models.

### Example 6

### Vascular Engineering Reactor (VER) for Regenerative Medicine

Adequate vascularization of tissue structures that closely recapitulate human physiology is crucial for improving survival rate and function of tissue engineered constructs. The microscale technologies with hydrogel techniques have offer precise control over various aspects of these tissue constructs including fluid flow, chemical gradients, localized extracellular matrix and biomechanical and electrical chemical stimuli. These functional aspects of tissue constructs play a vital role for normal cardiac development and regulate cardiac functions through signal transduction pathways. In order to assess the potential functional tissue construct using induced pluripotent stem cells, the differentiated myocardium should display highly organized sarcomeres, cellular junctions, and an extracellular matrix surrounding the cardiac cells in 3-D cell culture. Therefore, there is an urgent clinical need to engineer functionally viable regenerative tissues using stress parameters that mimic the native environment. Such model systems with externally applied forces will further our understanding of therapeutic approaches to cardiac regeneration and enable to manufacture regenerative medicine. Therefore Biopico Systems Inc develops “Vascular Engineering Reactor (VER) for Regenerative Medicine” with the goal of manufacturing. This VER system will be validated in a GLP regulated environment for pre-clinical and subsequent clinical adaptation. The VER system will provide complementary features such as electro mechanical stimulations capabilities and electrophysiological monitoring in a fully automated fashion and would help in the development of functional tissues for drug testing, disease modeling tissue repair and regenerative medicine manufacturing. The VER system will be established as inexpensive, easily manipulated, easily reproducible, physiologically representative of human disease, and ethically sound system for regenerative medicine manufacturing. The global regenerative medicines market size is expected to reach USD 49.41 Billion by 2021, at a CAGR of 23.7% during the forecast period of 2016 to 2021. The VER system can contribute to this market by establishing an innovative functional tissue manufacturing platform that will stimulate and monitor cells in functional assay. Biomedical research has relied on systemic animal studies and convenient 2-d cell cultures for several decades. However, the studies fail to recapitulate human and so microphysiological systems have showed promise to mimic the structure and function of native tissues. However, keeping the tissues alive for weeks' using perfusion of media or nutrients with integrated sensors for insitu monitoring and electromechanical stimuli to achieve functional tissues have not been realized. Therefore we extend our expertise in perfusion fluidics and electromechanical stimulation and monitoring to manufacture functional cardiac tissue for regenerative medicine. The proposed Vascular Engineering Reactor (VER) platform uses multimaterial 3D printing of viscoelastic inks fabricate vascular channels for perfusion of media and integrated sensors for long-term functional stimulation and monitoring. A cell on bioreactors is an adaptive mechanical structure that both receives and responds to biochemical, biomechanical, and bioelectrical signals. Cardiac cells can be mechanically and electrically stimulated by tensile, compressive, or cyclic strain which influences a number of cellular phenomena. Such understanding of how cells respond to stimuli is a critical step in learning how to direct cells in vitro to develop regenerative tissues for cardiac applications. Multi-electrode array-based assay to monitor electrophysiological properties cardiac cells and their responses to potential functional is highly significant for regenerative medicine. The field potential spikes, firing rate measurements can predict the effect of stimuli on both repolarization (QT screening) and conduction properties of cardiomyoctytes. During continuous live-cell monitoring and analysis, cells are not disturbed by the observation and analysis and so repeated measures over time provide powerful insight into the time course of biology and provides greater control over critical assay conditions. Using such system, morphology of the cells, contraction ability, proliferation rate, presence of intercellular adhesion structures, organization of myofibrils, mitochondria morphology, endoplasmic reticulurn contents, cytoskeletal filaments and extracellular matrix distribution, and expression of markers of cardiac differentiation can be studied in order to characterize the VER system.

1. A method for cell and organ culture on standard well plates or custom well plates or channels, the method comprising:
loading cells or organs in to at least one of the plurality of wells or microwells;
closing the well plates using a microfluidic plate;
pumping media or reagents into or out of the wells with at least one of the plurality of fluidic channels and fluidic tips;
performing media exchange or perfusion of media for cell or organ culture in one of the plurality of wells or microwells from at least one of the plurality reservoirs or wells.

2. The method of claim 1, wherein recirculation of media is performed within a well or across plurality of wells through filters to remove any molecules or subcellular or cellular species or without any filters;

3. The method of claim 2, wherein recirculation of media is performed across at least in one of the plurality of organs or from one organ such as the heart to one of the plurality of organs describing human physiology.

4. The method of claim 1, wherein the fluidic, electrical or optical instrumentations are controlled by Bluetooth low energy communication and data or image acquisition of the cells from at least one of the plurality of well, is carried out using Wi-Fi communication while incubating for long term cell culture or drug study.

5. The method of claim 1, wherein cells are cultured on at least one of the plurality of inserts or gels or scaffold within a well plate with fluidic exchange ports in inserts.

6. The method of claim 5, wherein cells are cultured on electrodes within an insert with porous substrates to exchange medium across top and bottom chambers.

7. The method of claim 1, wherein the microfluidic plates are connected with electrical reader plate to acquire data from field potential signal electrodes or impedance electrodes or transepithelial electrical resistance electrodes.

8. The method of claim 1, wherein a set of closed wells or fluidic channels for 3-d gel based cell culture for vascularization is connected to perfusion system.

9. The method of media or reagent exchange or perfusion is achieved by pushing the fluid from a reservoir into at least one or plurality of wells using an air pump and pulling the fluid into a reservoir from at least one or plurality of well using a vacuum pump through valves with plurality of ways connection.

10. The method of claim 9, wherein backflow or pressure balance is accomplished by incorporating additional vacuum or air pumps to provide positive or negative pressure at the reservoir

11. A multilayer fluidic plate comprising:
at least one or plurality of isolated sets of fluidic channels in at least one or plurality of layers;
at least one or plurality of inlets and outlet fluidic tips to pull or drop fluid into the well;
at least one or plurality of array of inlet and outlet ports to connect to a manifold;
at least one or plurality of channels connect from inlet or outlet ports to inlet or outlet fluidic tips.

12. The device of claim 11 wherein at least one or plurality of electrical connection circuit layer with electrical contacts.

13. The device of claim 11 wherein at least one or plurality of holes or windows for introducing probes for measurements or optical imaging.

14. A fluidic manifold comprising:
a top plate to run on a spring loaded hinge with constant or increasing thickness from the hinge side;
a bottom plate connected to the hinge to press the top plate;
a latch hinges on the bottom plate to lock the top plate through a locking bump on the top plate.

15. The device of claim 14 wherein the bottom side of the top plate having a set of pillars to press ports of microfluidic plate with the bottom plate.

16. The device of claim 14 wherein the bottom plate having holes or pockets to accommodate tubings that connect to reservoirs or pumps.

17. A method for recirculation and discrete perfusion for a well can be carried out by a set of two pumps and three way valves such that:
the pumps and valves are connected in series with inlet and outlet in to the well for recirculation with the valves connected to a particular way or direction;
the pumps and valves are connected in parallel to their corresponding fresh or used reservoirs in order to pump into or out of the well in succession with the valves connected to the other way or direction.

18. A method of claim 1 wherein gases such as oxygen and carbon-dioxide can be sent through additional channels in the microfluidic plate.

19. A method for multiple concentrations of drug or reagents solutions with a buffer solution can be carried out by using a plurality of pumps in multiple steps comprising:
controlling the proportional timings of the pumps;
alternate fluidic pulsing of the pumps for homogeneous mixing of the solutions;
discrete percentage of combinational fluids are produced by a pattern of fluid pulses with the appearance of each fluid segment spacing apart.

20. A method of claim 1 wherein additional electrical and mechanical stimulations are applied to cells or organs cultured on a cantilever plate where electromagnetic solenoid actuators apply mechanical pulses between two metallic posts and electrical stimulations are applied at the metallic posts.
